(19) United States
(12) Patent Application Publication
(10) Pub. No.: US 2016/0331539 A1
(43) Pub. Date: Nov. 17, 2016

(54) MODIFIABLE IMPLANTS

(71) Applicant: Elwha LLC, Bellevue, WA (US)

(72) Inventors: Roderick A. Hyde, Redmond, WA (US); Muriel Y. Ishikawa, Livermore, CA (US); Robert Langer, Newton, MA (US); Eric C. Leuthardt, St. Louis, MO (US); Stephen L. Malaska, Redmond, WA (US); Lowell L. Wood, JR., Bellevue, WA (US)

(21) Appl. No.: 14/710,458

(22) Filed: May 12, 2015

(51) Int. Cl. A61F 2/30

(52) U.S. Cl. CPC A61F 2/30; A61F 2002/30072; A61F 2002/30537; A61F 2002/30668; A61F 2250/0001; A61F 2250/0004

(57) ABSTRACT

Embodiments disclosed herein are directed to implants having a modifiable structural connectivity between one or more members thereof. In an embodiment, a modifiable implant includes a first member coupled to a second member by a reactive composite material having a release mechanism therein. A structural connectivity of the implant can be modified upon activation of the release mechanism. Systems and methods of using the same are disclosed.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority application(s)). In addition, the present application is related to the “Related Applications,” if any, listed below.

### PRIORITY APPLICATIONS

None

### RELATED APPLICATIONS

None

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

If the listings of applications provided above are inconsistent with the listings provided via an ADS, it is the intent of the Applicant to claim priority to each application that appears in the Priority applications section of the ADS and to each application that appears in the Priority applications section of this application.

All subject matter of the Priority applications and the Related applications and of any and all parent, grandparent, great-grandparent, etc. applications of the Priority applications and the Related applications, including any priority claims, is incorporated herein by reference to the extent such subject matter is not inconsistent herewith.

### SUMMARY

Embodiments disclosed herein are directed to implants having a modifiable structural connectivity between one or more members thereof. In an embodiment, a modifiable implant is disclosed. The modifiable implant includes a first member and a second member. The modifiable implant includes a reactive composite material coupling at least a portion of the first member to the at least a portion of the second the second member to limit movement therebetween. The reactive composite material includes a release mechanism configured to modify a structural connectivity of the modifiable implant upon activation of the release mechanism.

In an embodiment, a method of modifying an implant is disclosed. The method includes positioning an implant in a subject. The implant includes a first member having a first proximal portion and a first distal portion and a second member having a second proximal portion and a second distal portion. The implant includes a reactive composite material including a release mechanism therein. The reactive composite material couples at least the first distal portion to the second proximal portion. The method includes activating the release mechanism to at least partially dissociate the reactive composite material between the first member and the second member.

In an embodiment, a system for modifying an implant is disclosed. The system includes an implant having a first member; a second member; and a reactive composite material coupling at least a portion of the first member to at least a portion of the second member to limit movement therebetween. The reactive composite material includes a release mechanism therein. The system further includes a stimulus source configured to provide a stimulus to the release mechanism effective to cause activation thereof.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A-1D are cross-sectional views of reactive composite materials according to embodiments, which can be incorporated into any of modifiable implant disclosed herein.

FIGS. 2A and 2B are schematic cross-sectional views of modifiable implants according to embodiments.

FIGS. 3A and 3B are schematic cross-sectional views of an embodiment of a release mechanism including a cross-sectional view of a reactive composite material before and after use, which can be employed in any of the modifiable implants disclosed herein.

FIGS. 4A and 4B are schematic cross-sectional views of an embodiment of a release mechanism including a cross-sectional view of a reactive composite material before and after use, which can be employed in any of the modifiable implants disclosed herein.

FIG. 4C is a schematic diagram of a release mechanism according to an embodiment.

FIG. 5 is a schematic cross-sectional view of an embodiment of a release mechanism including a cross-sectional view of a reactive composite material before use, which can be employed in any of the modifiable implants disclosed herein.

FIGS. 6A-6F are isometric cut-cutaway views of modifiable implants according to embodiments.

FIG. 7 is a schematic diagram of a modifiable implant including more than one reactive composite material and release mechanism according to an embodiment.

FIG. 8A is a partial isometric view of a modifiable implant according to an embodiment.

FIG. 8B is a side view of a portion of the modifiable implant of FIG. 8A.

FIG. 9 is schematic diagram of a system for modifying an implant according to an embodiment.

FIG. 10 is a flow diagram of a method of modifying an implant according to an embodiment.

### DETAILED DESCRIPTION

Embodiments disclosed herein are directed to modifiable implants including at least one reactive composite material (RCM). Implants can include dental implants, joint replacements, support structure implants for bones, partial bone replacements, jaw implants, or pacemakers or other implants configured to aid in regulation of biological functions, among others. Subjects for such implants can include humans and animals alike. Implants can be used to repair damaged tissue, but require invasive surgeries for implantation, adjustment, or removal. In certain instances, a limited lifespan of the implant, growth, or even a reaction to an implant can take place, requiring removal or adjustment of the implant. Such surgeries can be fraught with complications including potential surgery related concerns, such as difficulty in removing or adjusting an implant that has been implanted in a subject or the trauma caused to an embedding tissue.

An implant can include one or more structural members. For example, an implant can include an artificial knee joint having a first member (e.g., lower leg portion of joint) that is rotatable about a second member (e.g., upper leg portion of joint). In implants including a plurality of members, one or more of a coating, packaging, an adhesive, or other component for maintaining the plurality of members as one integral unit can be employed. However, as the subject heals, an altered structural connectivity (e.g., flexibility or connection) between the plurality of members of the implant may be desired. A system including a modifiable implant can be used to alter a structural connectivity of the implant. A system for modifying the structural connectivity of an implant can include an implant having one or more members. The one or more members can be structurally connected in such a manner as to enable the one or more members to function as a single joined unit. The one or more members can be joined by one or more of adhesion (e.g., a contact adhesive between members), a joint between the one or more members, or a coating or covering extending about at least a portion of the one or more members (e.g., an RCM spanning from a portion of the first member to a portion of the second member).

After implantation, the structural connectivity of the implant can be altered (e.g., in situ) using a release mechanism associated with the RCM. Such structural connectivity can include one or more of an interface between the one or more members, freedom of movement between the members of an implant, the interface between tissue of the subject and the implant, or the structural rigidity of at least one member of the implant. Upon triggering the release mechanism, the RCM can be at least partially dissociated (e.g., dissolved, reacted, melted, or otherwise at least partially separated) between one or more members of the implant. Such dissociation can provide for a change in the structural connectivity of the implant (e.g., increased flexibility between the members, increased rotatability between members of the implant, separation of the members of the implant, etc.).

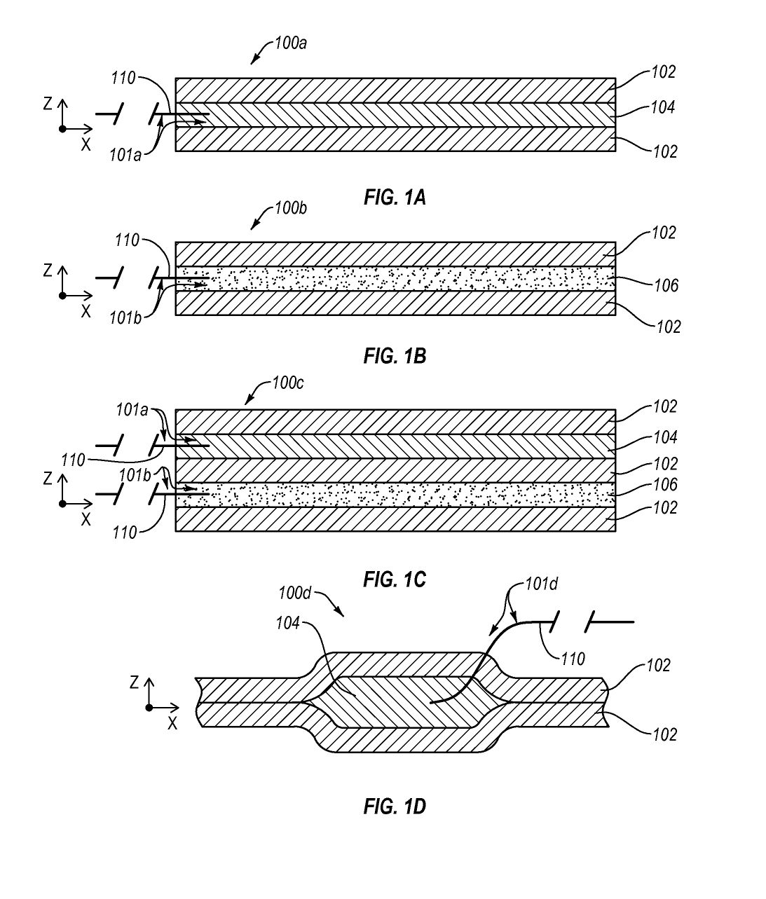

FIGS. 1A-1D are cross-sectional views of RCMs according to embodiments, which can be incorporated into any of the modifiable implants disclosed herein. RCMs can include one or more layers therein that can include differing materials, in one or more adjacent layers. RCMs can have multiple layers including one or more of reactive foil layers having nanometer or greater thickness that can be referred to herein as “nanofoil”, chemical agents, protective layers, compartments, or resistive material. For example, an RCM can include one or more (e.g., many thousands) layers of reactive nanofoil with a release mechanism associate therewith, such as between the one or more layers. The release mechanism can include a layer of material configured to react with the reactive nanofoil upon activation of the release mechanism. The release mechanism can be configured to initiate a chemical or thermal reaction in one or more components (e.g., between two or more components) of the RCM upon activation of the release mechanism. Once initiated (e.g., at one end, or at one corner), the chemical or thermal reaction can self-propagate throughout the RCM, traveling away from the initiation site throughout the entire volume of the RCM.

FIG. 1A is a cross-sectional view of a portion of RCM 100a having an electrical release mechanism 101a therein. The portion of RCM 100a includes two layers of reactive nanofoil 102. The reactive nanofoil 102 can include reactive materials, such as powders or metals. The powders or metals can be layered in raw powder or metal form or can be incorporated or impregnated in a binder material such as a polymer, an alloy, a ceramic, or an epoxy. The reactive nanofoil 102 can be produced with a suitable thickness via tape casting, chemical vapor deposition (CVD) deposition, or any other suitable technique. The reactive nanofoils 102 can include alternating layers of materials configured to react with one another or react with an adjacent material (e.g., a portion of a release mechanism). The reactive nanofoils 102 can include discrete portions of one or more materials disposed in a second material in a continuous or discontinuous sheet or pattern. Each individual layer of the reactive nanofoil 102 can be about 1 nm thick or more, such as about 1 nm to about 1 μm, about 5 nm to about 500 nm, about 10 nm to about 200 nm, about 20 nm to about 100 nm, or less than about 500 nm. In an embodiment, individual layers can exhibit the same or differing thicknesses from adjacent layers. In an embodiment, the reactive nanofoil 102 can include substantially only one layer. The reactive nanofoils 102 can include one or more of reactive metals, metal oxides, carbides, nitrides, Al, Ag, Au, B, Ba, Br, C, Ca, Ce, Cl, Cr, Co, Fe, Hf, Mg, Mn, Mo, Nb, Ni, Pd, Rh, Si, Ta, Ti, Th, W, V, Zr, Zn Fe₂O₃, Cu₂O, MoO₃, FeCo, FeCoOₓ, alloys (e.g., Monel® or Inconel®), a metallic glass, a ceramic, or a cermet. For example, reactive nanofoils can include NanoFoil™, commercially available from Indium Corporation, comprising alternating nanoscale layers of nickel and aluminum. Other combinations of materials which can be used to form reactive nanofoils are described in U.S. Pat. No. 6,736,942, which is incorporated herein by this reference in its entirety. These reactive nanofoils can include Rh/Si, Ni/Si, Zr/Si, Ni/Al, Ti/Al, Zr/Al, Ti/B, Ti/C, Al/Fe₂O₃, and Al/Cu2O. Other nanofoil compositions are described in “Self-Propagating Reactions in Multilayer Materials,” by T. P. Weihs in Handbook of Thin Film Process Technology, 1997, which is incorporated herein by this reference in its entirety. In an embodiment, the reactive nanofoils 102 can include a gel or foam (e.g., a hardsetting foam) in one or more layers therein. The gel or foam may be configured to react with or carry one or more reactive nanofoil materials, such as any noted above. Reactive nanofoils 102 can include a thickness of about 1 nm or more such as about 1 nm to about 1 μm, about 5 nm to about 500 nm, about 20 nm to about 300 nm, about 100 nm to about 600 nm, or about 50 nm or more.

A resistive material 104 or member can be disposed between one or more layers of the reactive nanofoil 102. In some embodiments, the resistive material 104 can include one or more layers of reactive nanofoil 102 itself (e.g., one or more Al layers of a Ni/Al or Ti/Al nanofoil). The resistive material 104 can include a material configured to provide resistance to electrical current, such as from an electrical connection 110, and thereby heat up upon an electrical current passing therethrough. The resistive material 104 can include a material configured to undergo a reaction with an adjacent material (e.g., reactive nanofoil) or self-react upon reaching a temperature effective to initiate such a reaction. For example, the resistive material 104 can include transition metals, alkaline earth metals, or alkali metals. For example, a suitable resistive material can include aluminum, nickel, iron, copper, zinc, tungsten, or silver.

Upon application of current to the resistive material 104, the resistive material 104 can build up heat, which can cause one or both of the resistive material 104 or the reactive nanofoil 102 to melt or chemically react. In some embodiments, once resistive material 104 builds up sufficient heat, it can initiate a self-propagating chemical reaction between the materials of reactive nanofoil 102. Such melting or reaction can result in at least a portion of the RCM changing physical states, such as from a solid phase to a liquid phase or gas phase. In some embodiments, the RCM includes a separate phase change material (e.g., not one of the components of the nanofoil) which is configured to absorb thermal energy released from the exothermic chemical reaction of the components of reactive nanofoil 102, to increase in temperature, and to undergo a phase change (e.g., from solid to liquid, solid to gas, or solid to liquid to gas). In some embodiments, the phase change material is disposed as one or more layers substantially parallel to layers of reactive nanofoil 102; layers of the phase change material can be interspersed with layers of reactive nanofoil 102, or can be outside reactive nanofoil 102 but still proximate to reactive nanofoil 102. In an embodiment, the energy release from the chemical reaction of the components of reactive nanofoil 102, (i.e., M_{NF} gms/area at H_{comb} J/gm) can provide sufficient energy to vaporize M_{PCM} gms/area of phase change material at Hvap J/gm, provided that M_{NF}H_{comb}>M_{PCM}Hᵥₐₚ. In some embodiments, the phase change material includes a material with low melting or vaporization temperature and/or with low heat of fusion or heat of vaporization. The phase change material may include a plastic (e.g., polyethylene, polycarbonate) or a metal (e.g., sodium, potassium, indium, or gallium). The phase change material may include a gallium, indium, or bismuth alloy (e.g., Indalloy available from Indium Corporation).

The material make-up or dimension (e.g., thickness) of the reactive nanofoil 102 can vary depending on one or more of desired mechanical properties of the RCM or implant on which the RCM is disposed (e.g., structural stability that the RCM provides to the one or more members of the implant), type or quantity of release mechanisms (e.g., type or quantity of resistive material or chemical release member) associated therewith, desired exothermic effect of the reaction of the reactive nanofoil on the surrounding tissue or implant, the number of layers (e.g., of reactive nanofoil, phase change material, protective layers, or release mechanisms) desired in the RCM, or any other suitable criteria. In an embodiment, the RCM on one or more members of an implant can be configured to enable at least a portion of one or more members to release from tissue in which the implant is embedded upon activation of the release mechanism.

As discussed in more detail below, a dimension (e.g., thickness or lateral dimension) of the release mechanism can vary depending one or more of the desired mechanical properties of the RCM or implant on which the RCM is disposed; type or quantity of release mechanisms; type, properties, thickness, or quantity of layers therein (e.g., reactive nanofoil layers); desired exothermic effect of the reaction of the reactive nanofoil on the surrounding tissue or implant; or any other suitable criteria. One or more of the dimensions of the RCM can be selected based upon one or more of the above mentioned criteria. For example, a lateral width (e.g., an X-axis dimension) of the RCM can be selected to wrap around the circumference of an implant a specified number of times. As another example, a lateral height (e.g., a Y-axis dimension) of the RCM can be selected to extend over a joint between two members of an implant and overlap onto a portion of each member by a selected distance. Suitable lateral widths or heights can be 1 mm or more, such as about 1 mm to about 50 cm, about 2 mm to about 25 cm, about 5 mm to about 10 cm, about 50 mm to about 25 mm, about 20 mm to about 5 cm, about 25 mm to about 125 mm, about 25 mm, about 10 mm, 1 mm, about 1 cm, about 2 cm, about 5 cm, about 10 cm, or greater than about 25 mm.

The thickness (e.g., a Z-axis dimension) of an RCM can be about 20 nm or more, such as about 20 nm to about 1 mm, about 40 nm to about 500 μm, about 100 nm to about 250 μm, about 500 nm to about 100 μm, about 50 nm to about 500 nm, about 500 nm to about 500 μm, about 25 μm or more, about 200 μm or more, about 100 nm, about 250 nm, about 500 nm, about 5 μm, about 40 μm, about 100 μm, about 200 μm, or about 1 mm or more.

FIG. 1B is a cross-sectional view of a portion of RCM 100b having at least a portion of release mechanism 101b therein. The release mechanism 101b can include a chemical release member 106 therein. The portion of RCM 100a includes two layers of reactive nanofoil 102. Examples of reactive nanofoil materials can include any of those disclosed above. At least one chemical release member 106 can be disposed at one site of reactive nanofoil 102, or between one or more layers of the reactive nanofoil 102. The chemical release member 106 can include a chemical agent or material configured to initiate a self-reaction and/or chemical reaction with one or more adjacent layers (e.g., nanofoil and chemical agent), upon receiving a stimulus such as electrical current from an electrical connection 110. In an embodiment, the stimulus can release the chemical agent from a compartment into contact with either another chemical agent or with reactive nanofoil 102. In an embodiment, the stimulus can heat one or more components of the chemical agent so as to initiate combustion between them. The resultant combustion energy can then thermally couple to the reactive nanofoil 102, initiating combustion between layers thereof. In an embodiment, the self-reacting chemical agent may include a reactive nanofoil. For example, the RCM may compose a relatively larger portion of one composition of nanofoil coupled in one or more sites to a smaller portion of another reactive nanofoil, serving as chemical release member 106. The resulting reaction between the at least one chemical release member 106 and the reactive nanofoil 102 can result in at least partial dissociation (e.g., dissolution, degradation, or melting) of the RCM 100b. The at least one chemical release member 106 can include a material configured to undergo a reaction with an adjacent material (e.g., reactive nanofoil) or self-react upon reaching a temperature effective to initiate such a reaction. For example, suitable chemical agents or materials can include one or more of reactive metals, metal oxides, carbides, nitrides, Al, B, Ba, Br, C, Ca, Ce, Cl, Cr, Co, Fe, Hf, Mg, Mn, Mo, Nb, Ni, Pd, Rh, Si, Ta, Ti, Th, W, V, Zr, Zn Fe₂O₃, Cu₂O, MoO₃, FeCo, FeCoOₓ, alloys (e.g., Monel® or Inconel®), a metallic glass, a ceramic, a cermet, or any other chemical compound configured to react with a material in an RCM. The chemical agent 106 can be in liquid form (e.g., H₂O₂), in powdered form, in solid form (e.g., reactive nanofoil), in gel form, in foam form, incorporated into a binder material or matrix, or incorporated into an alloy or a ceramic.

Upon application of stimulus (e.g., electrical current) to the chemical release member 106, the chemical agents therein can react, which can cause one or both of the chemical agent or the reactive nanofoil 102 to melt, or chemically react. Such melting or chemical reaction can result in at least a portion of the RCM becoming liquid or gaseous.

Multiple stacked layers of RCMs according to any embodiment herein, or single RCMs having multiple layers of any of the components of RCMs disclosed herein—collectively referred to as RCM stacks—can be used to change a structural configuration of an implant. FIG. 1C is a cross-sectional view of a portion of an RCM stack 100c having an electrical release mechanism 101a and a (chemical) release mechanism 101b therein. The RCM stack 100c can include at least three layers of reactive nanofoil 102. As depicted, a first layer can be disposed on a first side of the first release mechanism, which can be similar or identical to electrical release mechanism 101a. A second layer of reactive nanofoil 102 can be positioned one a second side of the release mechanism 101a. A second release mechanism can be positioned adjacent to the second layer of reactive nanofoil. For example, the second release mechanism can be configured similar or identical to release mechanism 101b, including a chemical release member 106. A first side of second release mechanism 101b can be positioned adjacent to the second layer of the reactive nanofoil 102, such as one the side opposite the first release mechanism 101a. The resistive material 104 and/or chemical release member 106 can include any of those respective materials disclosed above. A third layer of reactive nanofoil 102 can be positioned on the second side of the second release mechanism. In some embodiments, the RCM can include one or more release mechanisms therein, such as about two to about 10 release mechanisms, about two release mechanisms, about three release mechanisms, or about 5 release mechanisms or more.

In an embodiment, RCMs can be layered over one another to form an RCM stack having a plurality of RCM layers therein. The number of the RCM layers can be increased or decreased to produce a selected thickness of the RCM stack. A respective RCM stack can include more than one of any of the RCMs disclosed herein. For example, an RCM stack can include a plurality of layers each including the RCM 100a shown in FIG. 1A and having the release mechanism 101a. In an embodiment, an RCM stack can include a plurality of layers including the RCM 100a shown in FIG. 1A including the release mechanism 101a and the RCM 100b shown in FIG. 1B including the release mechanism 101b. The thickness of the RCM stack can vary depending on the desired mechanical strength of the RCM stack, the materials in the RCMs therein, the implant type, or other suitable criteria. An adhesive layer (not shown) can be present between any of the adjacent layers of the RCM stack. The thickness of an RCM stack can be about 50 nm or more such as about 50 nm to about 200 μm, about 100 nm to about 100 μm, about 1 μm to about 150 μm, about 250 nm to about 50 μm, about 50 nm to about 500 nm, about 500 nm to about 1 μm, about 200 μm or less, about 100 nm, about 250 nm, about 500 nm, or less than 1 mm. RCM stacks can be used interchangeably with the RCMs in any of the embodiments herein.

In some embodiments, multiple layers of reactive nanofoil can be overlaid upon each other. Each layer can be different to an adjacent layer, such that the adjacent layers are configured to react with one another upon receiving a sufficient stimulus, such as from a release mechanism. In an embodiment, each layer of reactive nanofoil can be substantially similar or identical to each adjacent layer, wherein a chemical reaction substantially throughout the layers is initiated by one or more release mechanisms therein.

FIG. 1D is a cross-sectional view of a portion of RCM 100d having at least a portion of an electrical release mechanism 101d therein. In an embodiment, release mechanisms can be positioned across an entire lateral dimension (e.g., in the X and Y directions) of an RCM, or can be disposed in discrete portions the RCM, extending less than the entire lateral dimension of an RCM, such as in a band, a pocket, or pattern (e.g., continuous or discontinuous patterns). As shown in FIG. 1D, two layers of reactive nanofoil 102 can be substantially in contact with each other throughout an RCM. At one or more intermediate points therein, the RCM 100d can include one or more release mechanisms. The release mechanism 101d, configured as an electrical release mechanism substantially similar or identical to the electrical release mechanism 101a, can be disposed within a discrete lateral portion of the RCM 100d, such that activation of the release mechanism 101d can affect regions of the RCM 100d adjacent to the release mechanism 101d. This discrete lateral portion can be positioned adjacent to a portion of an implant wherein increased compliance (e.g., increased flexibility or rotation) is desired after implantation. Upon activation of the release mechanism, portions of the RCM adjacent to the release mechanism can dissociate leaving other more distant portions of the RCM substantially unaffected. A selected portion of an RCM (e.g., portion surrounding the joint of an implant) can be selectively removed or otherwise dissociated via such an embodiment.

In some embodiments, a release mechanism can be disposed within a discrete lateral portion of the RCM. The reactive nanofoil can be configured to dissociate across the entire lateral dimensions thereof responsive to a reaction with or caused by the activated release mechanism in only a portion of the RCM. Thus, in some embodiments, only a portion of the RCM can include the release mechanism capable of causing substantially the entire RCM to at least partially dissociate. For example, an RCM can include a release mechanism associated therewith having a release member disposed therein in a checkerboard pattern or a linear pattern.

In an embodiment, at least a portion of the release mechanism can occupy about 100% of the lateral area of the RCM. In one or more embodiments, at least a portion of the release mechanism can occupy less than 100% of the lateral area of the RCM, such as 90% or less of the lateral area, about 90% to about 5%, about 75% to about 25%, about 60% to about 40%, about 50% to about 10%, about 20% to about 5%, about 10%, about 25%, or about 50% of the lateral surface area of the RCM. In an embodiment, at least a portion of the release mechanism can extend across an entire a lateral dimension of the RCM. For example, the release mechanism can extend horizontally across (e.g., in the X-axis direction) an RCM in a small band occupying about 10% of the lateral height (e.g., the Y-axis direction) of the RCM. In some embodiments, at least a portion of the release mechanism can occupy a discrete pocket, pattern (e.g., continuous or discontinuous pattern), or isolated lateral portion of the RCM. Such embodiments can promote structural rigidity yet allow a change in structural connectivity via dissociation of only a small portion of the total RCM.

In an embodiment, the RCM can include one or more protective layers configured to reduce or eliminate effects of the release mechanism from penetrating into adjacent tissue or the implant. For example, a protective layer can be configured to protect adjacent tissue from the chemical or thermal effects (e.g., increased temperature) of a reaction between the reactive nanofoil and the release mechanism.

In such an embodiment, a protective layer can include an endothermic reactant configured to react with one or more of the reactive nanofoil, the release mechanism, or the products of a reaction therebetween to cause an endothermic or neutralizing reaction therewith. The endothermic or neutralizing reaction can limit the extent of heat from an exothermic reaction or extent of damaging chemical reactants (e.g., acidic or basic chemical species) caused by activation of the release mechanism. Such an embodiment can provide an enthalpy of reaction with relation to the surrounding tissue and/or implant as near to zero as possible.

In an embodiment, the protective layer can include one or more chemical reactants configured to react with one or more of the reactive nanofoil, the release mechanism, or the products of a reaction therebetween to neutralize the chemical components thereof to at least limit toxic or corrosive chemicals from damaging tissue or the implant. The protective layer can include one or more compounds or molecules embedded within a substrate such as a polymer, epoxy, ceramic, metal alloy, or cermet. Suitable endothermic reactants can include inorganic or organic reactants such as hydrated barium hydroxide, alumina trihydrate, ammonium chloride, nitrates, thiocyanate, thionyl chloride and cobalt(II) sulfate heptahydrate, or any other suitable reactant configured to react with the chemical agent, nanofoil, or reaction products thereof.

The protective layer can be configured such that the protective layer also at least partially dissociates upon activation of the release mechanism. The thickness or material of the protective layer can be selected based upon one or more of the type of the release mechanism or reactive nanofoil, the size (e.g., thickness) of the release mechanism or reactive nanofoil, the heat expected to be generated by the release mechanism, the type material of the implant, or the type of tissue in which the implant is deployed.

In an embodiment, an RCM can include a protective layer between the reactive nanofoil and the tissue of a subject. The protective layer can exhibit a thickness sufficient to limit effects of the release mechanism on surrounding tissue, such as the resistive material heating up, reaction of the release mechanism and/or reactive nanofoil, or any other release mechanism effects. In an embodiment, the thickness of the protective layer can be sufficient to provide enough of an endothermic reactant to react with the release mechanism and/or reactive nanofoil to substantially limit or eliminate the exothermic effects therefrom from damaging surrounding tissue. The thickness of the protective layer can also be selected to provide substantially only enough material in the protective layer to react with one or more of the release mechanism, the reactive nanofoil or products thereof, such that the protective layer is substantially entirely dissociated (e.g., dissolved or exhausted) upon use thereof.

In an embodiment, the protective layer can be disposed adjacent to the release mechanism. For example, in an embodiment where the release mechanism extends across only portion of a lateral dimension of the RCM, the protective layer can similarly extend over only a portion of a lateral dimension of the RCM, including only over the same portions as the release mechanism described above. In an embodiment, the protective layer can be disposed substantially parallel to or blanketing the portions of the RCM having the release mechanism therein. In an embodiment, the protective layer can be disposed substantially parallel or blanketing the portions of the RCM having the release mechanism therein and extend slightly past such portions by a distance to ensure limitation of negative effects from use of the release mechanism on surrounding environments. In an embodiment, the protective layer can cover (e.g., overlap) a larger lateral dimension of the RCM containing the release mechanism by 2% of the lateral dimension of the release mechanism or more, such as a 5% to about 50% larger lateral dimension, or covering a 10% larger lateral dimension.

The RCMs disclosed herein can be used to provide structural connectivity (e.g., connection, rigidity, etc.) between one or more members of an implant. Any of the RCMs described herein can be disposed in or on an implant to provide a modifiable structural connectivity to the implant, such as allowing increased motion of one member of an implant with respect to another member of the implant (e.g., rotation, axial movement, flexibility, lateral movement, bending, sliding, separation, etc.). An RCM can be disposed across, around, or in a joint between members of an implant, and the release mechanism causes the RCM to at least partially dissociate to thereby modify or enable modification of the structural connectivity of members of the implant.

In an embodiment, the reactive nanofoil of an RCM can be configured to undergo self-reaction between the constituents thereof responsive to a stimulus (e.g., electrical, thermal, acoustic, etc.) such that a resistive material or chemical release member is not required to at least partially dissociate the RCM. In such embodiments, an operable connection between the stimulus source (e.g., electrical connection to a capacitor) and one or more layers of reactive nanofoil can be included in the release mechanism to initiate reaction of the RCM. In such embodiments, the RCM can include substantially only reactive nanofoil layers of one or more compositions.

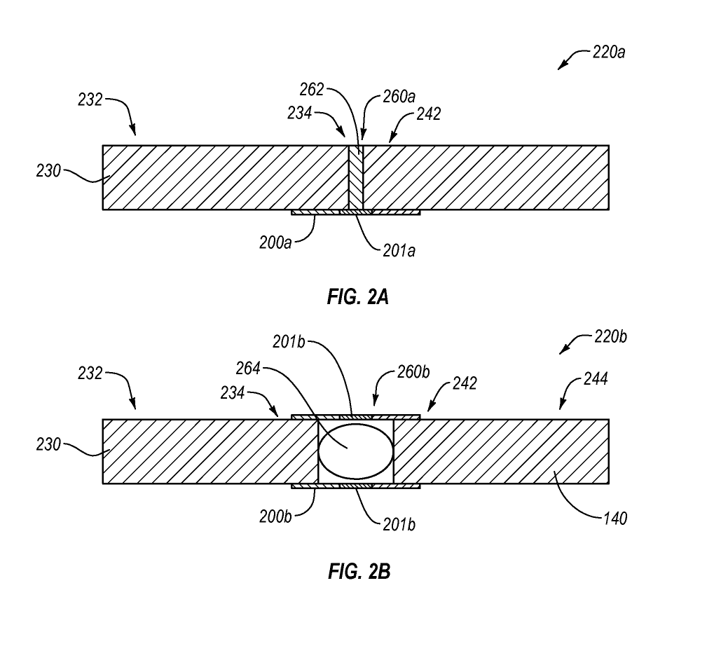

FIG. 2A is a schematic cross-sectional view of a modifiable implant 220a according to an embodiment. The implant 220a can include one or more members, a joint therebetween, or an RCM therebetween. The one or more members can include a first member 230, a second member 240 and, in some embodiments, additional members (not shown) depending on the particular implant design. The first member 230 of the implant can include a proximal portion 232 and a distal portion 234 (e.g., first proximal portion and first distal portion), and the second member 240 can include a proximal portion 242 and a distal portion 244 (e.g., second proximal portion and second distal portion). The distal portion 234 is positioned substantially adjacent to the proximal portion 242. The implant 220a can include a joint 260a between the first and second members 230 and 240. The joint 260a can include one or more of a mechanical connection (e.g., a hinge, a ball joint, a slip joint, a universal joint, etc.), an adhesive connection, or any other suitable connection between adjacent members of an implant. For example, as shown, the joint 260a can include an adhesive 262 disposed in a space between the two members 230 and 240, and the adhesive 262 secures the members together until it is broken or dissolved. The thickness of the adhesive 262 or distance between the members can vary depending on the type of adhesive required, the clearance required for the members to move with respect to each other upon dissociation of the adhesive, or the structural strength (e.g., bending or shear strength) desired in the implant prior to use of the release mechanism. Suitable adhesive thicknesses include 10 μm or more, such as about 10 μm to about 1 cm, about 100 μm to about 1 mm, about 500 μm, or more than about 100 μm.

The first member 230, the second member 240, and the joint 260a can be configured as a biological joint replacement including portions of the each of the biological members (e.g., bones) on opposite sides of the joint 260a. For example, in an embodiment, the first member 230 can be configured as a distal end of a femur, the second member 240 can be configured as a proximal end of a tibia, and the joint 260a can be configured as the artificial equivalent of the knee joint therebetween. In an embodiment, the first member 230 can be configured as a distal end of a proximal phalanx, the second member 240 can be configured as a proximal end of a middle phalanx, and the joint 260a can be configured as the artificial equivalent of the metacarpophalangeal joint therebetween. Further embodiments, of modifiable implants can include hip joints, elbow joints, vertebral joints, ankle or wrist joints, or any other suitable joint. The modifiable implants disclosed herein can also be used in non-biological environments, such as folding or rotating mechanical members, telescoping mechanical members, or other suitable non-biological environments wherein a modifiable structural connectivity is desired.

The modifiable implant 220a can include at least one RCM 200a extending from one member to another member. The at least one RCM 200a can extend about at least a portion of one or more members. For example, as shown, the RCM 200a can extend from a lateral portion of the first member 230 to a lateral portion of the second member 240 about a portion of the lateral surface thereof, thereby at least partially connecting the first and second members 230 and 240. The RCM 200a can provide a desired structural connectivity between the one or more members of the implant 220a. For example, the RCM 200a can provide a structural connection between the members to limit bending, rotation, shear, separation, lateral movement, axial movement, or other relative movement between the members.

The RCM 200a can be secured to one or more of the first member or the second member by a mechanical fastener (e.g., staples, screws, etc.), an adhesive (e.g., non-toxic medical adhesive or glue) layer or spot, compression (e.g., shrink fit), a weld, stitching, a retaining member thereabout, or other suitable attachment technique. In an embodiment, the RCM 200a can be disposed between the members of an implant, such as interposed therebetween. An adhesive layer can be disposed between the RCM 200a and at least one of the members of the implant (e.g., first distal portion or the second proximal portion).

As shown in FIG. 2A, the at least one RCM 200a can include a release mechanism 201a associated therewith, such as in or on the at least one RCM 200a. The release mechanism 201a can be configured according to any of the release mechanisms disclosed herein, including one or more of the type of release mechanism, one or more of the dimensions of the release mechanism, or amount of release mechanisms. For example, the release mechanism 201a can be disposed about at least a portion of the RCM 200a on the implant adjacent to the joint 260a, such as between the one or more members of the implant. Upon activation of the release mechanism 201a, the at least one RCM 200a can at least partially dissociate in the area adjacent to the release mechanism 201a, thereby providing an altered structural connectivity between the one or more members 230 and 240, such as increased flexibility, rotatability, or relative movement therebetween. For example, the joint 260a, the first and second members 230 and 240, or the release mechanism 201a can be configures such that activation of the release mechanism 201a can permit axial or lateral motion along an interface between the members 230 and 240, such as between the distal portion 234 and the proximal portion 242.

FIG. 2B is a schematic cross-sectional view of a modifiable implant 220b according to an embodiment. The modifiable implant 220b or portions thereof can be similar or identical to the modifiable implant 220a or portions thereof, with like portions having like numbering. The modifiable implant 220b includes the first member 230, the second member 240, and a joint 260b therebetween. The joint 260b can include a mechanical connection 264, such as a hinge, a ball joint, a slip joint, a universal joint, an artificial joint (e.g., knee joint or finger joint), or any other suitable connection configured to enable relative movement between the members 230 and 240. For example, the joint 260b can include a hinge configured to enable relative angular motion between the first and second members 230 and 240 about the joint 260b. It may be desirable to restrict the movement of the joint 260b for a time, such as during implantation or healing. The modifiable implant 220b can also include an RCM 200b configured to modifiably limit the structural connectivity of the first and second members 230 and 240 with respect to the joint 260b or each other.

The first member 230, the second member 240, or the joint 260b can be configured as a biological joint replacement substantially as described above.

In an embodiment, the RCM 200b can extend across less than an entire dimension or surface of one or more of the members 230 or 240. In an embodiment, the RCM 200b can extend entirely around a dimension of one or more members (e.g., circumferentially or fully encapsulating). For example, as shown in FIG. 2B, the RCM 200b can extend around an entire lateral or circumferential dimension of the first member 230 and the second member 240. Such embodiments can impart a desired structural connectivity to the modifiable implant, while simultaneously covering the joint 260b and any adhesive 262 or mechanical connection 264 between the first and second members 230 and 240. For example, as shown, the RCM 200b can extend from a distal portion of the first member 230 to a proximal portion of the second member 240 circumferentially about the entire lateral surface of each, thereby connecting the first and second members 230 and 240 across the joint 260b. The RCM 200b can provide a structural connection between the members or resist bending, rotation, shear, separation, or other relative movement between the members.

The RCM 200b can be secured to one or more of the first member 230 or the second member 240 by a mechanical fastener, an adhesive, compression, a weld, stitching, a retaining member thereabout, or other suitable attachment means.

The at least one RCM 200b further includes a release mechanism 201b associated therewith, such as in or on the at least one RCM 200b. The release mechanism 201b can be configured according to any of the release mechanisms disclosed herein, including one or more of the type of release mechanism, one or more of the dimensions of the release mechanism, or amount of release mechanisms. For example, the release mechanism 201b can be disposed about at least a portion of the RCM 200b on the modifiable implant adjacent to the joint 260b, such as between one or more members of the modifiable implant. The release mechanism 201b can extend about a portion of the RCM 200b, such as an area of the RCM 200b between the first and second members 230 and 240. Upon activation of the release mechanism 201b, the at least one RCM 200a can at least partially dissociate (e.g., completely dissolve or disintegrate between the first and second members) in an area adjacent to the release mechanism 201b, thereby providing an altered structural connectivity between the first and second members 230 and 240, such as increased flexibility, rotatability, or relative movement therebetween.

In an embodiment, the joint can include both an adhesive and a mechanical connection therein. Activation of the RCM can cause the adhesive therein to be exposed, enabling the adhesive to break away or chemically react with surrounding environment to dissolve, thereby enabling the mechanical connection increased movement such as, increased flexibility, increased rotation, or freedom of movement.

In an embodiment, the RCM on one or more members of an implant can be configured to enable at least a portion of one or more members to release from tissue in which the implant is embedded upon activation of the release mechanism. The one or more members can release from the embedding tissue by substantially completely dissociating the RCM, thereby freeing any tissue (e.g., scar tissue, muscle, or bone) formerly connected thereto of the connection to the dissociated RCM, and by extension, the implant. In an embodiment, the RCM can be configured to enable at least one of the first and second members to release from an embedding tissue upon activation of the release mechanism by at least reducing an lateral dimension of the at least one of the first or second members sufficient to enable withdrawal from an embedding tissue.

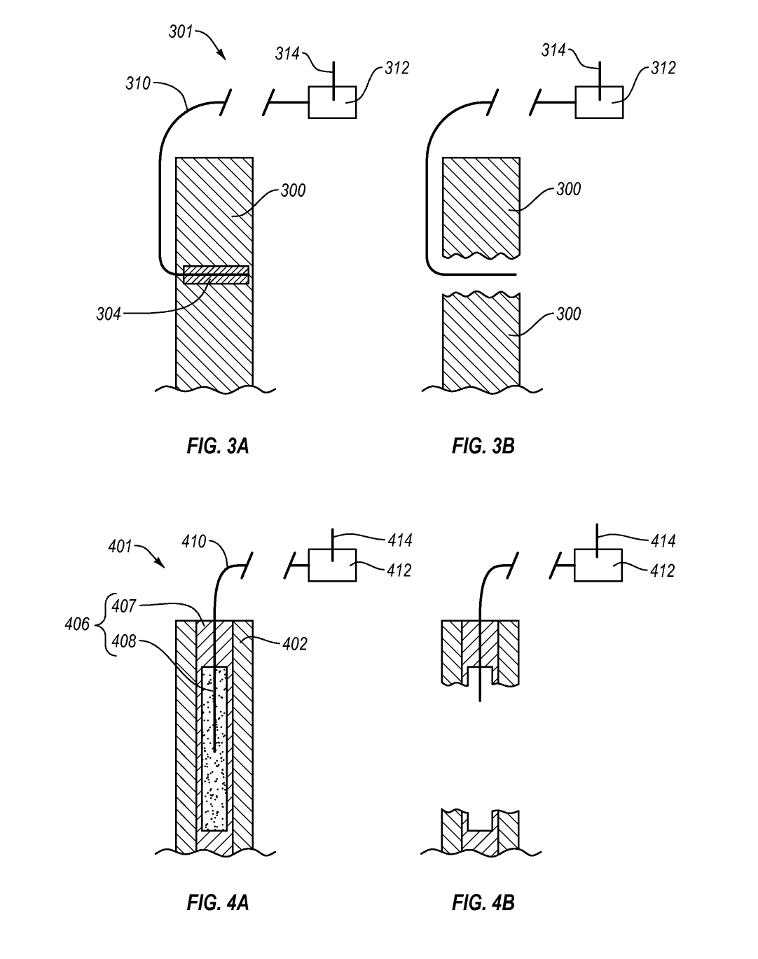

FIGS. 3A and 3B are schematic cross-sectional views of an embodiment of a release mechanism including cross-sectional views of an RCM before and after use. FIG. 3A depicts an RCM 300 having a release mechanism 301 therein, which can be employed in any of the modifiable implants disclosed herein. The release mechanism 301 includes a resistive material 304, a capacitor 312, an electrical connection 310 therebetween, and an antenna 314 operably coupled to the capacitor 312 in a circuit. The circuit can include a resonator (not shown) therein. In an embodiment, the capacitor 312 can be configured to be charged via electromagnetic energy (e.g., radio frequency energy directed to the antenna 314, resonator, or a battery) and discharged through the resistive material 304 operably coupled to the capacitor 312. In an embodiment, the capacitor 312 can be configured to be charged by ultrasonic vibrations or infrared light directed to the antenna 314, resonator, or battery.

The RCM 300 can be configured identical or similar to any of the RCMs disclosed herein, such as RCMs 100a-100d. The resistive material can be similar or identical to the resistive material 104. For example, the resistive material 304 can be disposed between one or more layers of the reactive nanofoil in the RCM 300. For example, the resistive material 304 can be disposed at one site, an edge, or a corner of the reactive nanofoil in the RCM 300. The resistive material 304 can provide resistance to electrical current such as from an electrical connection 310, and thereby heat up upon receiving electrical current. The resistive material 304 can include a material configured to undergo a reaction with an adjacent material (e.g., reactive nanofoil) or self-react upon reaching a temperature effective to initiate such a reaction.

The capacitor 312 can include an implantable capacitor having a sufficiently small size to be associated with one or more portions of the modifiable implant. For example, suitable capacitors can include miniaturized ceramic or electrolytic capacitors such as those made and sold under the TAZ series name by AVX Corporation of Fountain Inn, S.C. The capacitor size or capacitance can be selected based upon one or more of the size of the implant, the size of the RCM 300, the type of RCM 300, the type of resistive material 304, or the type of chemical agent or material. The capacitor 312 can be configured with a 1 nF capacitance or greater, such as about 0.001 μF to about 1000 μF, about 0.01 μF to about 100 μF, or about 0.1 μF to about 10 μF. The capacitor 312 can be configured to deliver a voltage of 0.1 V or more, such as about 1 V to about 1000 V, about 4 V to about 100 V, about 10 V to about 50 V, about 5 V, about 20 V, or less than about 10 V. The capacitor 312 is configured to discharge through the resistive material 304.

The capacitor 312 can be connected to the resistive material 304 by the one or more electrical connections 310. The one or more electrical connections 310 can be of sufficient length to enable the capacitor 312 to be remote from the resistive material 304 or RCM 300, such as in tissue of a subject, in or on a member of the implant, or external to the subject. The electrical connection 310 can be at least about 100 μm long, such as about 1 mm to about 20 cm, about 5 mm to about 10 cm, about 10 mm to about 2 cm, less than 10 cm, or greater than about 10 mm long. The one or more electrical connections 310 can be a wire of any suitable material configured to efficiently conduct electrical current therethrough. The electrical connection 310 can include an insulating material over at least a portion thereof. The insulating material can prevent voltage loss to the external environment such as the subject tissue or implant. In an embodiment, the electrical connection 312 can include a copper wire connected to the resistive material 304 extending away from the RCM 300 to a capacitor 312 remote from the RCM 300. The wire can be coated with an insulator thereabout except for portions thereof in contact with or within the resistive material 304 or the capacitor 312.

The antenna 314 can include a pin antenna, monopole antenna, a dipole antenna, a resonator (explained in more detail below), or any other structure capable of harvesting electromagnetic radiation (e.g., radio frequency radiation), sonic vibrations, or light to produce an electrical current. The antenna 314 can extend away from the capacitor 312, electrical connection 310, or RCM 300. The antenna 314 can be at least partially integrated into a structure of one or more of the circuit, capacitor 312, electrical connection 310, or RCM 300. For example, the antenna 314 can be disposed in at least a portion of one or more of the capacitor 312, electrical connection 310, or RCM 300. The antenna 314 can be tuned to a particular frequency or wavelength, such that the release mechanism is not accidentally activated. The release mechanism 301 can also include one or more rectifiers (not shown) between the antenna 314 and the capacitor 312 to convert alternating current to direct current. Thus, in an embodiment the release mechanism 301 can be triggered by a small electrical pulse.

In some embodiments, the release mechanism can include a circuit having one or more resonators therein, such as in addition to an antenna. The one or more resonators can be configured to receive narrow-band radiofrequency radiation. The one or more resonators can collect the narrow-band radio frequency radiation and convert the radiofrequency radiation into electrical current or generate a higher voltage or current than is received therein. Suitable resonators can include MEMS devices such as MEMS resonators or miniaturized RLC circuits.

In an embodiment, a release mechanism can include a circuit having a capacitor and a battery. The capacitor is configured to be charged via the battery (e.g., slowly charged) and discharged (e.g., rapidly discharged) through a resistive material or member operably coupled to the capacitor. Thus, in an embodiment the release mechanism can be triggered by a small electrical pulse.

As shown in FIG. 3A, the resistive material 304 can be positioned at an intermediate point of the RCM 300 perpendicularly therethrough (e.g., perpendicular to the direction of the layers of the RCM 300). In an embodiment, the resistive material 304 can be positioned or extend laterally therethrough (e.g., parallel to the direction of the layers of the RCM 300), at a discrete location (e.g., a substantially spherical or polygonal body) therein, or any other position suitable to enable the resistive material 304 to react with one or more components of the RCM 300 (e.g., continuous or discontinuous patterns).

As shown in FIG. 3B, upon activation of the release mechanism 301, such as by application of voltage from the capacitor 312, the RCM 300 can be at least partially dissociated. The electrical connection 310 can remain after dissociation of the RCM 300. However, in an embodiment, the portion of the electrical connection 310 in contact with the resistive material 304 can also at least partially dissociate. In an embodiment, only the portion of the RCM adjacent to the resistive material 304 can react therewith or otherwise dissociate (e.g., melt). A gap in the RCM 300 can be observed in the immediate area the resistive material 304 occupied prior to activation.

In an embodiment, the RCM 300 can be configured such that the resistive material 304 extends across substantially the entire lateral dimensions of the RCM 300. In such embodiments, the release mechanism 301 can include one or more electrical connections 310 therein to ensure satisfactory dissociation of the RCM 300. In such embodiments, substantially the entire RCM 300 can be dissociated (e.g., dissolved, reacted, or melted). For example, the RCM 300 can be melted or reacted at least partially from a solid state to a liquid state or at least partially from a solid state to a gaseous state.

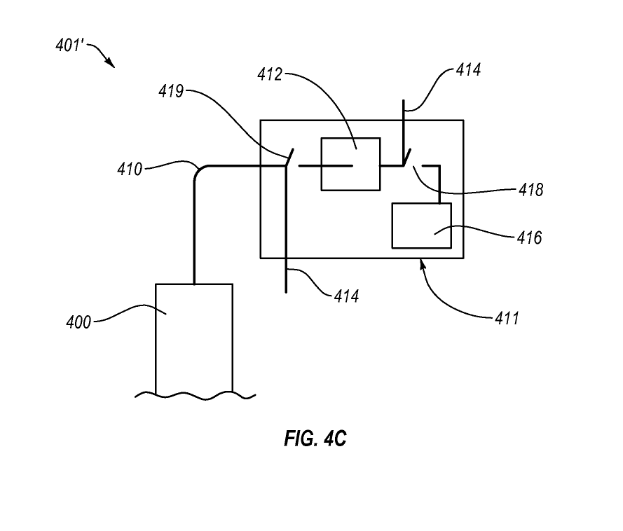

FIGS. 4A and 4B are schematic cross-sectional views of an embodiment of a release mechanism including cross-sectional views of an RCM before and after use, which can be employed in any of the modifiable implants disclosed herein. FIG. 4A depicts an RCM 400 having a release mechanism 401 therein, which can be employed in any of the modifiable implants disclosed herein. The release mechanism 401 includes at least one chemical release member 406, a capacitor 412, an electrical connection 410 therebetween, and an antenna 414 operably coupled to the capacitor 412. The capacitor 412, electrical connection 410, or antenna 414 can be identical or similar to the respective capacitor 312, electrical connection 310, or antenna 314. The RCM 400 can be configured identical or similar to any of the RCMs disclosed herein, such as RCMs 100a-100d. The at least one chemical release member 406 can be similar or identical to the chemical release member 106. For example, the chemical release member 406 can be disposed between one or more layers of the reactive nanofoil 402 in the RCM 400. The chemical release member 406 can include a chemical agent or material configured to react or initiate a reaction responsive to electrical current such as from an electrical connection 410. In an embodiment, the chemical release member 406 can include a chemical agent or material configured to react or initiate a reaction responsive to ultrasonic vibration (e.g., indirectly through an antenna or capacitor triggered by ultrasound signals or directly by about 20 kHz or greater ultrasound signals), microwave radiation, or light (e.g., directly or indirectly via infrared light shone through tissue). The chemical release member 406 can include a material configured to provide resistance to electrical current and thereby heat up upon receiving electrical current. The chemical release member 406 can include a material configured to undergo a reaction with an adjacent material (e.g., reactive nanofoil) or self-react upon reaching a temperature effective to initiate such a reaction. The product of such a self-reaction can interact (e.g., at least partially melt, sublimate, or react) with the reactive nanofoil to at least partially dissociate the RCM 400.

The RCM 400 can include a first layer of reactive nanofoil 402, a second layer of reactive nanofoil 402, and a chemical release member 406 therebetween. In an embodiment, the chemical release member 406 can include a compartment 407 including a chemical agent 408 therein. The compartment 407 can include one or more walls completely enclosing the chemical agent 408 therein. The compartment 407 can include a material configured to remain stable until receipt of a stimulus (e.g., electrical current from the electrical connection 410). The material of the compartment 407 can be configured to melt, react, or otherwise interact with the chemical agent 408 upon receipt of a stimulus, sufficient to at least partially dissociate the RCM 400 adjacent thereto. In an embodiment, only a portion of the compartment 407 can be configured to respond to receipt of the stimulus, while a second (e.g., larger) portion remains inert. Upon reaction of the chemical agent 408 (e.g., with the material of the compartment 407 or at least partial melting the material of the compartment 407), one or more of the chemical agent 408, a product of the reaction of the chemical agent 408, the material of the compartment 407, or a product of the reaction of the material of the compartment 407 can react with the reactive nanofoil 402 adjacent thereto.

In an embodiment, the material of the compartment 407 can include inert or reactive components therein. The material of the compartment 407 an include transition metals, alkaline earth metals, alkali metals, organic compounds (e.g., polymers), inorganic compounds, ceramics, other suitable compounds, or mixtures of any of the foregoing. The chemical agent 408 can include a chemical or chemical compound configured to react upon stimulation, such as via electrical current from the electrical connection 410 or sonic vibration (e.g., ultrasound). In an embodiment, the stimulus can release the chemical agent 408 from the compartment 407 into contact with either another chemical agent or with reactive nanofoil 402. In an embodiment, the stimulus can heat one or more components of the chemical agent 408 so as to initiate combustion between them. The resultant combustion energy can then thermally couple to reactive nanofoil 402, initiating combustion between its layers. In an embodiment, the self-reacting chemical agent 408 may include a reactive nanofoil. For example, the RCM may compose a relatively larger portion of one composition of nanofoil coupled in one or more sites to a smaller portion of another reactive nanofoil, serving as chemical release member 406. The chemical agent 408 can include one or more of reactive metals, metal oxides, carbides, nitrides, Al, B, Ba, Br, C, Ca, Ce, Cl, Cr, Co, Fe, Hf, Mg, Mn, Mo, Nb, Ni, Pd, Pt, Rh, Si, Ta, Ti, Th, W, V, Zr, Zn Fe₂O₃, Cu₂O, MoO₃, FeCo, FeCoOₓ, alloys (e.g., Monel® or Inconel®), a metallic glass, a ceramic, a cermet, or any other chemical compound configured to react with one or more materials in an RCM (e.g., reactive nanofoil layer). The chemical agent 408 can be in liquid form (e.g., H₂O₂), in powdered form, in solid form (e.g., a reactive nanofoil), incorporated into a binder material or matrix, or incorporated into an alloy or a ceramic.

In an embodiment, the chemical release member 406 can be positioned or extend substantially perpendicularly or laterally therethrough, at a discrete location therein, or any other position suitable to enable the chemical release member 406 to react with one or more components of the RCM 400 (e.g., continuous or discontinuous patterns). As shown in FIG. 4B, upon activation of the release mechanism 401, such as by application of voltage from the capacitor 412, the RCM 400 can be at least partially dissociated. The electrical connection 410 can remain after dissociation of the RCM. However, in an embodiment, the portion of the electrical connection 410 in contact with the chemical release member 406 can also at least partially dissociate. In some embodiments, only the portion of the RCM adjacent to the chemical release member 406 can react therewith or otherwise dissociate (e.g., melt). A gap in the RCM 400 can be observed in the immediate area the chemical release member 406 formerly occupied, thereby separating portions of the RCM 400 from one another.

In an embodiment, the RCM 400 can be configured such that the chemical release member 406 extends about substantially the entire lateral dimensions of the RCM 400. In such embodiments, the release mechanism 401 can include one or more electrical connections 410 therein, to ensure satisfactory dissociation of the RCM 400. In such embodiments, substantially the entire RCM 400 can be dissociated (e.g., dissolved, reacted, or melted). For example, the RCM 400 can be at least partially melted or reacted (e.g., chemically or thermally) at least partially from a solid state to a liquid state, or at least partially from a solid state to a gas state.

FIG. 4C is a schematic diagram of a release mechanism 401′ according to an embodiment, which can be employed in any of the modifiable implants disclosed herein. The release mechanism 401′ includes a circuit 411 having a capacitor 412 and one or more batteries 416 operably coupled thereto via an electrical connection 410. The capacitor 412 can be configured to be charged via the battery 416 and discharged through the chemical release member or resistive material operably coupled to the capacitor 412. The capacitor 412 and the electrical connection 410 can be similar or identical to any capacitor or electrical connection described herein. The battery 416 can be configured such that it does not add unsatisfactory bulk or volume to the implant or RCM. For example, suitable batteries 416 can include a thin film battery, a button cell, a zinc-air cell (e.g., using oxygen from the water in surrounding tissues or fluids), or other suitable miniaturized batteries. A suitable thin film battery can include a flexible thin film lithium-ion battery, such as the LiTe*STAR™ thin-film rechargeable battery or Thinergy® battery by Infinite Power Solutions, or equivalents thereof. The battery 416 can be configured to deliver 1 V or more, such as about 1V to about 20 V, about 2 V to about 5 V, about 3 V, about 4 V, or about 10 V. The battery 326 can be configured to deliver 0.1 mA or more, such as about 0.1 mA to about 1 A, about 0.2 mA to about 0.5 mA, or about 1 A. The battery can be connected to the capacitor via a voltage enhancing circuit so as to charge the capacitor to a higher voltage than that of the battery.

The circuit 411 can further include an electrical switch 418 or gate between the battery 416 and the capacitor 412. The electrical switch 418 can be operably coupled to an antenna 414. The antenna 414 can be configured to receive a specific frequency or wavelength of electromagnetic radiation, ultrasonic vibrations, or infrared light. In an embodiment, the antenna 414 is coupled to a narrow-band resonator, configured to respond to a specific stimulus (e.g., specific type or frequency). For example, the electrical switch 418 can be configured to close upon receipt of an electrical stimulus from the antenna 414 responsive to the appropriate frequency or wavelength of electromagnetic radiation. The electrical switch 418 can include a MEMS switch, such as an RF switch, or a microwave switch by way of example. Once the electrical switch 418 closes, the battery 416 can charge the capacitor 412, and the capacitor 412 can discharge. In an embodiment, an electrical switch 419 can be located between the capacitor 412 and the resistive material or chemical release member, such that the capacitor 412 can only be discharged into the RCM 400 upon receipt of the appropriate frequency or wavelength of electromagnetic radiation. The electrical switch 419 can be similar or identical to the electrical switch 418.

In an embodiment, the release mechanism 401′ can include one or more of the electrical switches 418 or 419 described above. For example, the circuit 411′ can include the electrical switch 418 coupled to the antenna 414 and configured to receive a first radio frequency; and the electrical switch 419 can be coupled to an additional antenna 414 and configured to receive a second radio frequency. In such an embodiment, the capacitor 412 of the release mechanism 401′ can only be charged and discharged into the RCM 400 upon receipt of both radio frequencies. In an embodiment, a plurality of electrical switches 418 or 419 and associated antennas 414 can be configured to operate on the same radio frequency or differing radio frequencies. In an embodiment, the circuit 411′ can be disposed in or on an implant, such as in or on a member of the implant, or in or on the RCM. Suitable radio frequency radiation can include those used for radio, telephone, wireless telephone, or other suitable radio frequencies. In some embodiments, the release mechanisms (e.g., antennas or resonators) can be configured to receive an encrypted or narrow-band radio signal to limit the chance of accidental activation of the release mechanisms.

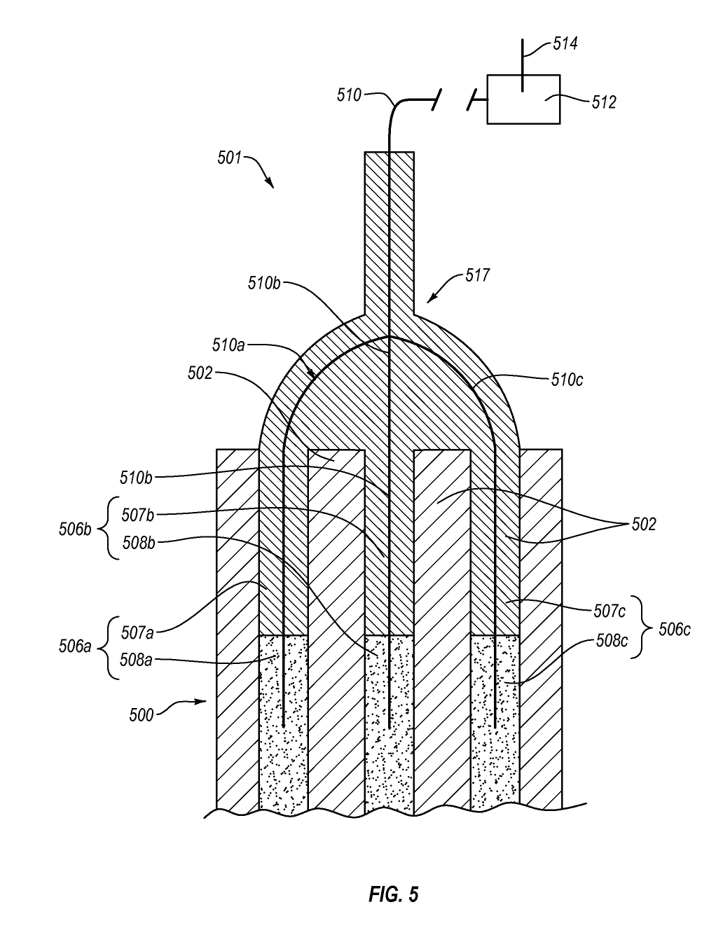

FIG. 5 is a schematic diagram of an embodiment of a release mechanism 501 including a cross-sectional view of an RCM 500 before use, which can be employed in any of the modifiable implants disclosed herein. The RCM 500 can include a plurality of reactive nanofoil layers 502 and a release mechanism 501 having one or more resistive materials or chemical release members 506a-506c therebetween. The release mechanism 501 can include a plurality of chemical release members 506a-506c, at least one capacitor 512, one or more electrical connections 510 therebetween, and an antenna 514 operably coupled to the at least one capacitor 512. The release mechanism 501 can include one or more electrical switches similar or identical to switches 418 or 419.

The capacitor 512, electrical connection 510, or antenna 414 can be identical or similar to the respective capacitor 312 or 412, electrical connection 310 or 410, or antenna 314 or 414. The RCM 500 or any sub-components thereof can be configured identical or similar to any of the RCMs or sub-components thereof disclosed herein, such as RCMs 100a-100d. One or more of the chemical release members 506a-506c or portions thereof can be similar or identical to the chemical release member 106 or 406 or portions thereof.

Each of the at least one chemical release members 506a-506c can be disposed between one or more layers of the reactive nanofoil 502 in the RCM 500. Each chemical release member 506a-506c can include a chemical agent or material configured to react or initiate a reaction responsive to electrical current such as from electrical connection 510. One or more of the chemical release members 506a-506c can be similar or identical to one or more of the adjacent chemical release member 506a-506c. One or more of the chemical release members 506a-506c can be different from one or more of the adjacent chemical release member 506a-506c, such as being located in a different lateral portion of the RCM 500 or including a different chemical agent or compartment material therein. One or more of the chemical release members 506a-506c can include a material configured to provide resistance to electrical current such as from an electrical connection 510, and thereby heat up upon receiving electrical current. One or more of the chemical release members 506a-506c can include a material configured to undergo a reaction with an adjacent material (e.g., reactive nanofoil) or self-react upon reaching a temperature effective to initiate such a reaction. The product of a self-reaction can interact (e.g., melt, sublimate, or thermally or chemically react) with the reactive nanofoil 502 to at least partially dissociate the RCM 500.

The at least one capacitor 512 can be operably coupled to one or more of at least one antenna 514 or at least one battery (not shown). The capacitor 512 can be operably coupled to the antenna 514 or battery by one or more electrical connections 510. The electrical connection 510 can extend to or through a manifold 517. In the manifold 517, the electrical connection 510 can split into a plurality of branches, such as electrical connections 510a-510c. In an embodiment, the electrical connection 510 can be a harness including a plurality of branches, such as electrical connections 510a-510c. In an embodiment, each electrical connection 510a-510c can be operably coupled to least one capacitor 512, such as each to the same or a different at least one capacitor 512.

Each electrical connection 510a-510c can be operably coupled to a respective resistive material, a material of a compartment 507a-507c, or a chemical agent 508a-508c. As shown, the electrical connection 510a can branch from electrical connection 510 in the manifold 517, extend into the compartment 507a, and into the chemical agent 508a therein, thereby forming a chemical release member 506a. The electrical connection 510b can branch from electrical connection 510 in the manifold 517, extend into the compartment 507b, and into the chemical agent 508b therein, thereby forming a chemical release member 506b. The electrical connection 510c can branch from electrical connection 510 in the manifold 517, extend into the compartment 507c, and into the chemical agent 508c therein, thereby forming a chemical release member 506c.

In an embodiment, the one or more chemical release members 506a-506c can be positioned in substantially the same lateral location (e.g., location between the nanofoil layers running parallel to the layers) in the RCM 500. For example, the one or more chemical release members 506a-506c can be positioned substantially throughout the entirety of one or more lateral dimensions of the RCM 500. In an embodiment, the one or more chemical release members 506a-506c can be positioned in substantially different lateral locations in the RCM 500, such as substantially parallel to the layers but each spaced by a lateral distance therebetween. For example, the chemical release member 506a can be disposed in a discrete distal portion of the RCM 500, the chemical release member 506b and be disposed in a medial portion of the RCM 500, and the chemical release member 506c can be disposed in a proximal portion of the RCM 500. Such a configuration can enable selective modification of the modifiable implant. For example, in an embodiment, a medial portion of the RCM 500 can be configured to restrict the relative movement between members around a joint more than a distal portion or a proximal portion. After implantation, it may be desired to allow only limited movement between members of the implant, such that activating the release mechanism disposed in one or more of the distal or proximal portion can be carried out. After a certain time has passed, such as enough time for healing or rehabilitation, it can desirable to further alter (e.g., increase) the range of relative movement between the members of the implant. Activation of a second release mechanism, such as the medially located release mechanism, can be carried out to further alter the structural connectivity of an implant.

In an embodiment, an electrical release mechanism, such as any described herein, can be used in place of one or more of the chemical release members 506a-506c. In an embodiment, the release mechanism 501 can include one or more of both of an electrical release mechanism or a chemical release member. One or more electrical release mechanisms, and any components thereof, can be disposed and positioned within the RCM 500 in a similar or identical way as the chemical release members 506a-506c.

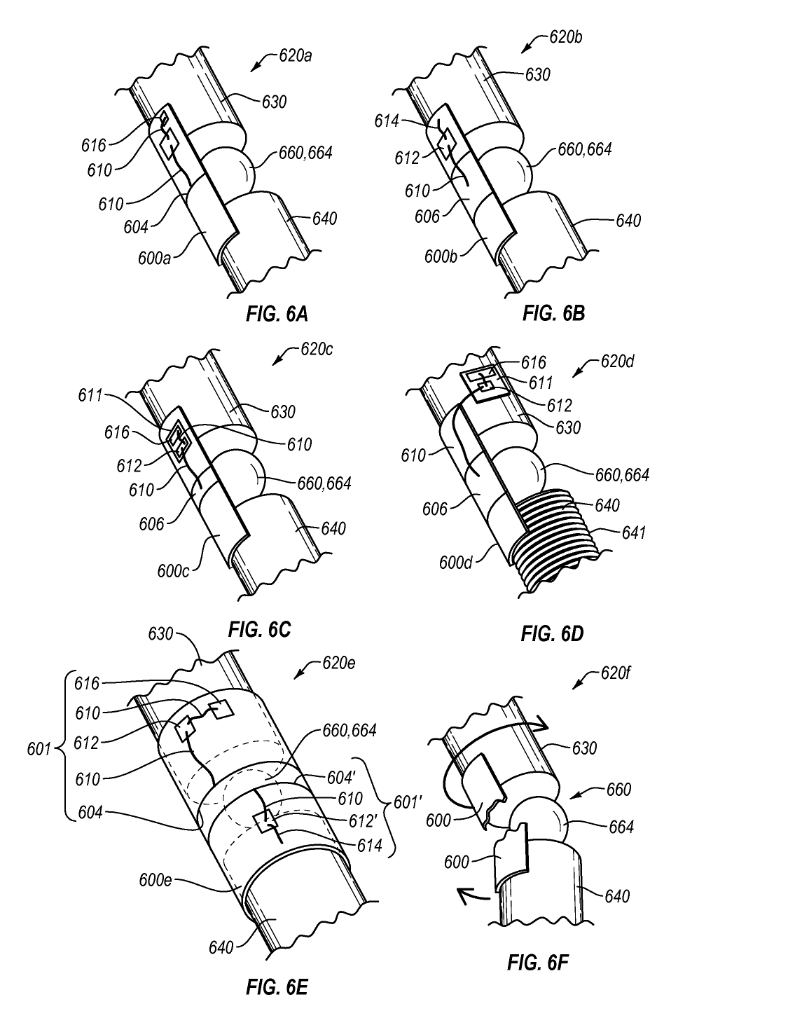

FIGS. 6A-6F are isometric cut-cutaway views of modifiable implants according to embodiments. FIG. 6A shows an implant 620a having a first member 630, a second member 640, a joint 660 therebetween having a mechanical connection 664. The implant 620a includes an RCM 600a extending between and extending about at least a portion of the first member 630 and at least a portion of the second member 640. The at least one RCM 600a or a component thereof can be configured or positioned similar or identical to any of the RCMs disclosed herein. The RCM 600a can include an electrical release mechanism including a resistive material 604 connected to a capacitor 612 by electrical connection 610. The capacitor 612 can be operably coupled to a battery 616 via electrical connection 610, effective to enable the battery 616 to charge the capacitor 612. The capacitor 612 can be discharged into the resistive material 604. The capacitor 612, battery 616, or electrical connection 610 can be similar or identical to any capacitor, battery, or electrical connection herein. For example, the capacitor 612 can be coupled to the battery 616 via the electrical connection 610, where the electrical connection includes a one or more electrical switches (not shown) therein. The one or more electrical switches can be similar or identical to any electrical switches disclosed herein.

The RCM 600a or the resistive material 604 can extend about only a portion of the members 630 or 640 of the modifiable implant 620a, such as about (e.g., circumferentially) only a portion of the outer surface of the members. For example, the RCM can extend about only a portion of a dimension of one or more of the first and second members 630 or 640, such as more than about 5% of the a lateral surface of one or more of the first and second members, about 5% to about 95%, about 10% to about 80%, about 25% to about 75%, about 40% to about 60%, about 20% to about 40%, about 50% to about 90%, about 25%, or about 50% of the a lateral surface of one or more of the first and second members. The resistive material 604 can include any desired lateral dimensions ranging from 1% of a lateral dimension of the RCM 600a to 100% of a lateral of the RCM 600a, such as about 1% or more, about 2% to about 90%, about 5% to about 80%, about 10% to about 75%, about 25%, to about 50%, about 5%, about 10%, about 20%, or less than about 90% of a lateral of the RCM 600a. The resistive material 604 can be disposed in any portion of the RCM 600a. As shown, the resistive material 604 can be positioned in the RCM at an intermediate point between the first and second members 630 and 640. The resistive material can be located equidistantly from both members, closer to one member, or at least partially overlap one or more members. The capacitor 612 or the battery 616 can be positioned on or in the RCM 600a. In an embodiment, one or more the capacitor 612 or battery 616 can be positioned external to the RCM 600a, such as on the first or second member 630 or 640, or can be positioned in tissue external to the modifiable implant 620a. In an embodiment, the modifiable implant 620a can include an antenna operably coupled to the capacitor 612.

FIG. 6B shows an implant 620b including an RCM 600b extending between and extending about at least a portion of the first member 630 and at least a portion of the second member 640. The at least one RCM 600b or a component thereof can be configured or positioned similar or identical to any of the RCMs disclosed herein. The RCM 600b can include a chemical release member 606 operably coupled to a capacitor 612 by an electrical connection 610. The capacitor 612 can be operably coupled to an antenna 614 configured to charge the capacitor 612 such that the capacitor can be discharged into the chemical release member 606. The antenna 614 can be similar or identical to the antenna 314 describe above or include any antenna capable of harvesting electromagnetic radiation (e.g., radio frequency radiation) to produce a current. In an embodiment, the modifiable implant 620b can include a battery (not shown) operably coupled to the capacitor 612. The capacitor 612, antenna 614, battery, or electrical connection 610 can be similar or identical to any capacitor, battery, antenna, or electrical connection herein. In an embodiment, the capacitor 612 can be coupled to the antenna 614 via the electrical connection 610, where the electrical connection includes one or more electrical switches (not shown) therein. The one or more electrical switches can be similar or identical to any electrical switches disclosed herein.

The RCM 600b or the chemical release member 606 can extend about only a portion of the members 630 or 640 of the modifiable implant 620b, such as about (e.g., circumferentially) only a portion of the outer surface of the members. For example, the RCM 600b can extend about only a portion of the outer surface of one or more of the first and second implants 630 or 640, such as those disclosed above with respect to RCM 600a. The chemical release member 606 can include any desired lateral dimensions including those disclosed above with respect to the resistive material 604 in FIG. 6A. The chemical release member 606 can be disposed in any portion of the RCM 600b such as any of those disclosed above with respect to the resistive material 604 in FIG. 6A. As shown, the chemical release member 606 can be positioned in the RCM at an intermediate point between the first and second members 630 and 640, such as substantially spanning the entire space between the first and second members or less. The chemical release member 606 can be located equidistantly from both members, closer to one member, or at least partially overlap one or more members. The capacitor 612, antenna 614, or a battery (not shown) can be positioned on or in the RCM 600b. In an embodiment, one or more of the capacitor 612, the antenna 614, or battery (not shown) can be positioned external to the RCM 600b, such as on the first or second member 630 or 640, or can be positioned in tissue external to the modifiable implant 620b.

FIG. 6C shows an implant 620c including an RCM 600c extending between and extending about at least a portion of the first member 630 and at least a portion of the second member 640. The at least one RCM 600c or a component thereof can be configured or positioned similar or identical to any of the RCMs disclosed herein, such as similar to RCM 600b. For example, the RCM 600c is substantially similar to the RCM 600b. The RCM 600c includes a circuit 611 having a battery 616 operably coupled to the capacitor 612 rather than the antenna 614 of RCM 600b. The RCM 600c can include a chemical release member 606 operably coupled to a capacitor 612 by electrical connection 610. The battery 616 is configured to charge the capacitor 612 such that the capacitor can be discharged into the chemical release member 606 through the electrical connection 610. The capacitor 612, battery 616, or electrical connection 610 can be similar or identical to any capacitor, battery, or electrical connection herein. For example, the circuit 611 can include one or more electrical switches (not shown) such as between the battery 616 and the capacitor 612 or the between the capacitor 612 and the chemical release member 606. The one or more electrical switches can be similar or identical to any electrical switches disclosed herein.

The RCM 600c or the chemical release member 606 can extend about only a portion of the members 630 or 640 of the modifiable implant 620b as described above with respect to RCM 600b or the chemical release member 606 therein. The chemical release member 606 can be positioned in the RCM 600c at an intermediate point between the first and second members 630 and 640, such as substantially spanning less than 50% of the space between the first and second members 630 and 640 or less. In an embodiment, one or more of the capacitor 612, battery 616, or antenna (not shown) can be positioned at least partially in; on; or external to the RCM 600c, such as on the first or second member 630 or 640, or in tissue external to the modifiable implant 620c.

FIG. 6D shows an implant 620d having a similar to construction to that of the implant 620c. The modifiable implant 620d includes similar or identical components to that of the modifiable implant 620c. For example, the RCM 600d is substantially similar to the RCM 600c, including a chemical release member 606. The modifiable implant 620d includes a circuit 611 having a battery 616 and capacitor 612 remote from the RCM 600d, such as in or on the first member 630 as shown. A circuit 611 or one or more components thereof can be disposed on the surface of the first member 630 or the second member 640. The circuit 611 or components thereof can be secured to the member of the modifiable implant 620d via an adhesive, a mechanical fastener, soldering, integral construction, or any other suitable attachment. The electrical connection 610 can extend from the capacitor 612 external to the RCM 600d, through the RCM 600d, to the chemical release member 606 of the release mechanism (or resistive material in other embodiments).

In an embodiment, at least one of the capacitor 612 or the battery 616 can be internal a member of the modifiable implant, such as internal to (e.g., enclosed in, or embedded in a surface of) the first or second members 630 or 640. In such an embodiment, the electrical connection 610 can extend from inside of the member in which the battery or capacitor is stored to the RCM 600d disposed on the one or more members of the modifiable implant.

The first and second members 630 and 640 can be similar or identical to those in the modifiable implant 620c. In an embodiment, the first and second members 630 and 640 can be configured differently than in modifiable implant 620c. For example, the first and second members 630 and 640 can be configured as a head and shank of a screw respectively. In an embodiment, one or more of the first and second members 630 or 640 can include a threading thereon, such as threads 641. The threads 641 can extend over a portion of the second member 640 or over the entire longitudinal dimension of the second member 640. In an embodiment, one or more of the first member 630 and the second member 640 can include one or more features extending therefrom, configured to anchor the first or second member into subject tissue. Such extending feature can include protrusions or recesses, such as flanges, rods, grooves, threads, islands, divots, or any other feature suitable to add texture to the surface of a member. When the RCM 600d is substantially dissociated by activation of the release mechanism, the first member 630 or head can be structurally removed from the second member 640 or shaft.

FIG. 6E shows an implant 620e including an RCM 600e extending between and about the entire circumference of the first member 630 and the second member 640. In an embodiment, the modifiable implant or RCM can include more than one release mechanism. For example, the RCM 600e can include two release mechanisms 601 or 601′. The release mechanisms 601 or 601′ or components thereof can be configured similar or identical to any release mechanism or component thereof disclosed herein (e.g., similarly numbered components). The release mechanism 601 can include the resistive material 604, the electrical connection 610 coupling the resistive material 604 to the capacitor 612. The capacitor 612 can be operably coupled to a battery 616 via another electrical connection 610. The battery 616 can charge the capacitor 612, which can discharge into the resistive material 604. The release mechanism 601 can include one or more electrical switches (not shown) including an antenna or a resonator (e.g., narrow-band resonator) coupled thereto and configured to close or open upon receipt of a particular wavelength or frequency of electromagnetic radiation (e.g., radio frequency), effective to initiate or terminate charging or discharging of the capacitor 612.

The release mechanism 601′ can include a second resistive material 604′, the electrical connection 610 coupling the second resistive material 604′ to the second capacitor 612′. The second capacitor 612′ can be operably coupled to an antenna 614 or resonator configured to charge the second capacitor 612′, such as via harvesting radio frequency radiation. The second capacitor 612′ can discharge into the second resistive material 604′. The release mechanism 601′ can include one or more electrical switches (not shown) including an antenna or resonator (e.g., narrow-band resonator) coupled thereto and configured to close or open the electrical switch upon receipt of a particular wavelength or frequency of electromagnetic radiation (e.g., radio frequency). Operation of the electrical switches can be effective to initiate or terminate charging or discharging of the second capacitor 612′. The particular wavelength or frequency of electromagnetic radiation that the antenna(s) 614 associated with the release mechanism 601 are configured to receive can be identical or different to the particular wavelength or frequency of electromagnetic radiation that the antenna(s) 614 associated with the release mechanism 601′ are configured to receive.

In an embodiment, the particular wavelength or frequency of electromagnetic radiation that the antenna(s) 614 or resonators of both the release mechanisms 601 and 601′ are configured to receive can be the same wherein upon receipt of said particular wavelength or frequency of electromagnetic radiation both release mechanisms can be activated.

In an embodiment, the particular wavelength or frequency of electromagnetic radiation that the antenna(s) 614 of release mechanisms 601 and 601′ are configured to receive can be different. In such an embodiment, the release mechanisms 601 or 601′ or portions thereof can be selectively activated or deactivated. For example, it may be desirable to activate only release mechanism 601, in such an instance, the particular wavelength or frequency of electromagnetic radiation that the antenna(s) 614 of the release mechanism 601 can be selectively generated from an external source sufficient to trigger the activation of the release mechanism 601. An antenna associated with release mechanism 601′ can be configured to respond to a different stimulus (e.g., different type, frequency or wavelength) and be similarly actuated using the different stimulus.

While the release mechanisms 601 and 601′ are both shown as having a resistive material 604 or 604′ therein, in some embodiments, one or more of the release mechanisms can use a chemical release member. While the release mechanisms 601 and 601′ are shown as having a battery 616 or antenna 614 for charging the capacitors therein, in some embodiments, one or both release mechanisms in an RCM can include a battery charged capacitor or an antenna charged capacitor.

FIG. 6F shows an implant 620f in which portions of an RCM 600f thereof disposed on a portion of the first member 630 and the second member 640 are at least partially dissociated from the first and second members 630 and 640. The RCM 600f can be an at least partially dissociated from any of the embodiments herein, such as RCM 600a-600e. The dissociation of the RCM 600f can enable the first member 630 or the second member 640 to rotate, slide, pivot, or otherwise move with respect to the second member 640, the first member 630, the joint 660, or the mechanical connection 664. In an embodiment, the joint 660 can include adhesive in addition to or in place of the mechanical connection 664.

While the first and second members 630 and 640 in FIGS. 6A-6F are depicted as linear shafts, the first and second members 630 and 640 can exhibit any geometric configurations encountered in implants (e.g., devices or artificial biological structures). For example, a first member can be a screw and a second member can be a pin. In an embodiment, the first member can be a ball of a joint and the second member can a socket of a joint. In an embodiment, the first member can be at least a portion of an artificial vertebral bone and the second member can be at least a portion of an adjacent artificial vertebral bone. In some embodiments, the implant can include more than two members; such that more than one joint is associated therewith.

In some embodiments, more than one RCM can be used, such as more than one RCM about a single joint, or a plurality of RCMs can be used on a plurality of joints, such as one or more RCMs about each joint of a plurality of joints.

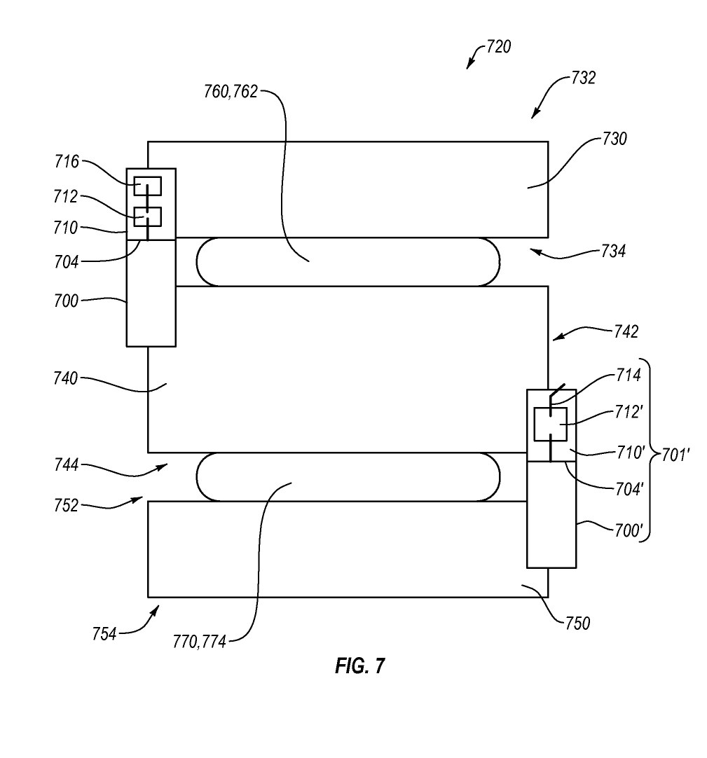

FIG. 7 is a schematic diagram of a modifiable implant 720 including more than one RCM and more than one release mechanism according to an embodiment. The modifiable implant 720 includes a first member 730 having a proximal end 732 and a distal end 734, a second member 740 having a proximal end 742 and a distal end 744, and a third member 750 having a proximal end 752 and a distal end 754. The modifiable implant 700 includes a first joint 760 between the first member 730 and the second member 740, and a second joint 770 between the second member 740 and the third member 760. While depicted as having a structure therein, the joints 760 or 770 can have no material or structure therein. Rather, the joint 760 or 770 can include an interface between the adjacent members of the modifiable implant 700. In an embodiment, the first joint 760 can include an adhesive material 762 therein, with the adhesive material bonding the first member 730 and the second member 740. In an embodiment, the second joint 770 can include an a mechanical connection 764 therein, with the mechanical connection bonding the second member 740 and the third member 750. One or more of the first joint 760 or the second joint 770 can include one or more of adhesive material or mechanical connection therein.

The modifiable implant 720 can include more than one RCM 700 or 700′, each extending about at least a portion of one or more of the members 730, 740, or 750 and across at least a portion of one or more of the joints 760 or 770. In an embodiment, the RCM 700 can extend about at least a portion of the first implant 730, across the joint 760, and about at least a portion of the second implant 740. In an embodiment, the RCM 700′ can extend about at least a portion of the second implant 740, across the joint 770, and about at least a portion of the second implant 750. The RCMs 700 or 700′ can be configured similar or identical to any RCM described herein. The RCMs 700 and 700′ can include the release mechanisms 701 and 701′ respectively. The release mechanisms 701 and 701′ can be similar or identical to any release mechanism disclosed herein, including any components thereof.

In an embodiment, the release mechanism 701 can include a resistive material 704 operably coupled to a capacitor 712 by an electrical connection 710. The resistive material 704 can be disposed about a circumferential dimension of the modifiable implant parallel to and within the joint 760 between the first and second members 730 and 740. The capacitor 712 can be operably coupled to a battery 716 by an electrical connection. The release mechanism 701 can include one or more electrical switches (not shown) operably coupled to an antenna (not shown) configured to activate the electrical switch upon receipt of a stimulus, such a radio frequency electromagnetic radiation. The release mechanism 701 can be configured to discharge electrical energy into the resistive material 704, effective to at least partially dissociate the RCM 700.

In an embodiment, the release mechanism 701′ can include a resistive material 704′ operably coupled to a capacitor 712′ by electrical connection 710′. The resistive material 704′ can be disposed about a circumferential dimension of the modifiable implant 720 parallel to and within the joint 770 between the second and third members 740 and 750. The capacitor 712 can be operably coupled to an antenna 714. The release mechanism 701 can be configured to discharge electrical energy into the resistive material 704, effective to at least partially dissociate the RCM 700. While depicted as electrical release mechanisms, one or more of the release mechanisms 701 or 701′ can include a chemical release member similar or identical to any chemical release member disclosed herein.

In an embodiment, the first member 730, the second member 740, the third member 750, the joint 760, and the joint 770 can be configured as artificial or actual bones or the joints therebetween, partial structures of the same, or support structures extending at least partially around the bones and joints of a subject. For example, the modifiable implant 720 can be configured as vertebrae and the joints therebetween; as fingers, including the bones and joints therebetween; as toes bones and the joints therebetween, or any other suitable multi jointed structure, either biological or otherwise.

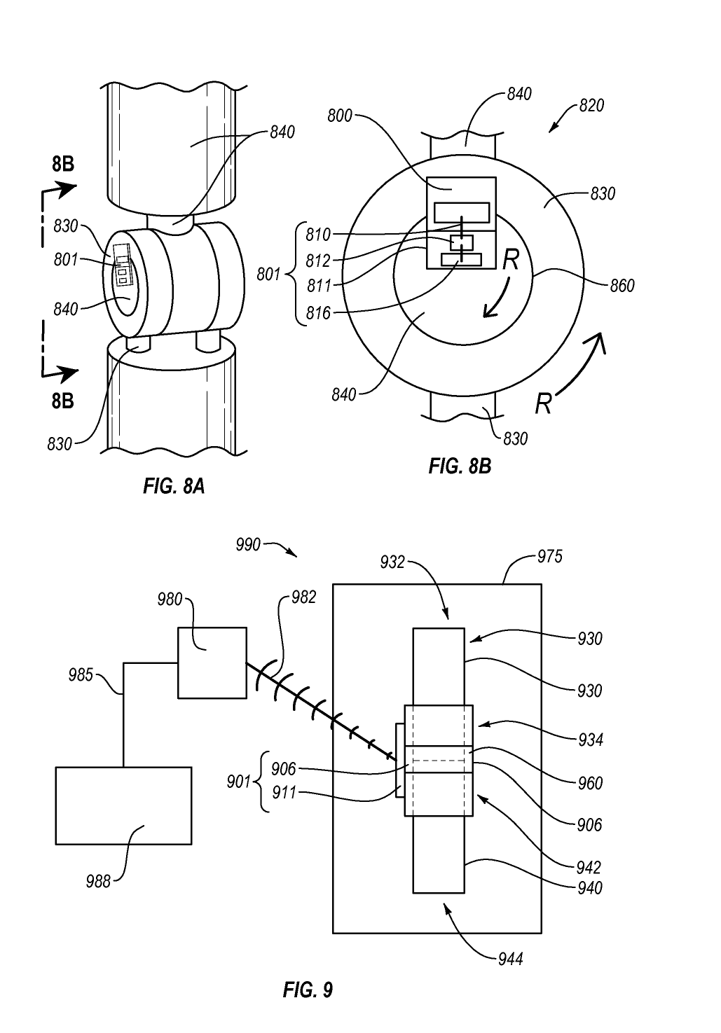

FIG. 8A is a partial isometric view of a modifiable implant 820 according to an embodiment. FIG. 8B is a partial side view of the modifiable implant of 820 along the line 8B. The modifiable implant 820 can include a first member 830 extending about (e.g., around) at least a portion of a second member 840. The members 830 and 840 can have a joint 860 therebetween. The joint 860 can be an interface between the first and second members 830 and 840. At least one RCM 800 can extend from at least a portion the first member 830 to at least a portion of the second member 840, thereby restricting rotational movement R therebetween. The RCM 800 or any components therein can be configured similar or identical to any of the RCMs described herein. For example, the RCM can include the release mechanism 801. The release mechanism 801 can include a chemical release member 806 operably coupled to a capacitor 812 by electrical connection 810. The capacitor 812 can be operably coupled to a battery 816 by an electrical connection to form a circuit 811. In an embodiment, the circuit 811 can include one or more antennas (not shown), resonators (not shown), or electrical switches (not shown) configured to selectively activate the release mechanism 801 upon receipt of a specific electromagnetic radiation. The chemical release member 806, the capacitor 812, the electrical connection, 810, the battery 816, one or more antennas, or one or more electrical switches can be similar or identical to any chemical release member, capacitor, electrical connection, battery, antenna, or electrical switch disclosed herein. In an embodiment, the release mechanism 801 can include a resistive material.

Upon activation of the release mechanism 801, the first member 830 and the second member 840 can rotate with respect to each other. In an embodiment, the modifiable implant 820 can be configured as an artificial knee or elbow joint.

In an embodiment, the circuit 811 can include one or more timers therein. The one or more timers can be configured to close a circuit between the battery 816 and the capacitor 812, sufficient to enable the capacitor 812 to charge, after a selected amount of time has elapsed. The one or more timers can be configured to close a circuit between capacitor 812 and the chemical releases member or resistive material, sufficient to enable the capacitor 812 to discharge therein, after a selected amount of time has elapsed. The one or more timers can be preprogrammed to cause an electrical switch to close after a selected time, such as based on a forecasted healing time or rehabilitation schedule (e.g., 1 day or more, 1 week, 1 month, or 1 month or more). In some embodiments, one or more timers can be included on any release mechanism disclosed herein.

In an embodiment, the circuit 811 can include a switch or circuit configured to open or close based upon receiving an encrypted signal. For example, a radio signal can be encrypted by frequency inversion. In some embodiments, digital encryption can be used. For example, an encrypted radio signal can be received by an antenna and transmitted to the switch which opens only upon receiving the encrypted signal. Such an embodiment can limit the chance of unintended activation of the release mechanisms herein.

FIG. 9 is schematic diagram of a system 990 for modifying an implant according to an embodiment. The system 990 can include one or more stimulus sources 980 and one or more modifiable implants 920. The modifiable implant 920 can be implanted within or on the subject 975. The modifiable implant 920 can be configured similarly or identical to any modifiable implant described herein, including any components thereof. In an embodiment, the modifiable implant 920 can include a first member 930 having a proximal portion 932 and a distal portion 934; a second member 940 having a proximal portion 942 and a distal portion 944; and a joint 960 between the distal portion 934 and the proximal portion 942. The joint 960 can be similar or identical to any joint disclosed herein. As shown, the joint 960 can include the interface of distal portion 934 of the first member 930 and the proximal portion 942 of the second member 940. The interface can include an adhesive or mechanical connection between the first and second members 930 and 940. The modifiable implant 920 can include an RCM 900 extending at least partially thereabout or therein. The RCM 900 can be configured similar or identical to any RCM disclosed therein, including any components thereof or any configurations thereof (e.g., layers, materials, stacks, etc.).

The RCM 900 can extend entirely around the lateral surface of a portion of the first and second members 930 and 940. The RCM 900 can include a release mechanism 901 similar or identical to any release mechanism disclosed herein. In an embodiment, the release mechanism 901 can include a chemical release member 906 operably coupled to a circuit 911 by an electrical connection 910. The circuit 911 can be configured similarly or identical to any circuit herein, such as including one or more of a capacitor, a battery, an antenna, one or more resonators, a timer, or electrical switches. The circuit 911 can include a capacitor and an electrical connection. The circuit 911 can include an antenna having or operably coupled to a resonator or the capacitor. The capacitor, electrical connection, antenna, resonator, or chemical release member 906 can be similar or identical to any disclosed herein. In an embodiment, the chemical release member can include a chemical agent (not shown) disposed between one or more layers of the RCM 900. The chemical agent can be in communication with the electrical connection 910, sufficient to enable an electrical current therethrough to trigger a reaction in the chemical agent. In an embodiment, the chemical release member 906 can include a chemical agent releasably stored in a compartment therein. The chemical agent can include a composition configured to degrade the RCM 900. The chemical release member 906 can include electrical release circuit 911 configured to emit an electrical charge effective to cause the compartment to rupture and release the chemical agent therein.

In an embodiment, the release mechanism 901 can include an electrical release mechanism including a circuit having a capacitor and an antenna or resonator. The capacitor can be configured to be charged via radio frequency energy directed to the antenna or resonator and discharged through a resistive material (not shown) operably coupled to the capacitor. The resistive material can heat up causing one or more layers (e.g., reactive nanofoil) of the RCM 900 to react and at least partially dissociate or degrade.

The stimulus source 980 can be configured to provide a stimulus 982 to the release mechanism 901 effective to cause actuation of the release mechanism 901. For example, the stimulus source 980 can be configured as an electromagnetic radiation generator, such as an radio frequency signal generator or a microwave generator; an electromagnetic field generator; or a sonic vibration (e.g., ultrasound or acoustic) generator. In an embodiment, the stimulus source 980 can be a radio frequency generator configured to send one or more specific frequencies (e.g., narrow-band frequency), amplitudes, or wavelengths of radio frequency radiation to one or more release mechanisms. The radio frequency generator can be configured to selectively send a specific frequency or wavelength depending on the desired modification to the structural connection of the modifiable implant. For example, a first radio frequency can trigger a first release mechanism or a portion thereof, and a second radio frequency can trigger a second release mechanism or a portion thereof. The effective range of the stimulus source 980 can depend on the type of stimulus 982, the size of the components of the release mechanism, or the type and location of the modifiable implant 920 within the subject 975. Effective ranges can include at least those ranges inside the same room, doctor's office, or medical facility.

In some embodiments, the stimulus source 980 can be operably coupled to a controller 988, such as a computer or tablet. The controller 988 can be configured to activate, direct, adjust, or provide instructions to the stimulus source 980. For example, the controller 988 can be a computer configured to control a characteristic (e.g., frequency, wavelength, duration, etc.) of the stimulus 982 generated by the stimulus source 980. The stimulus source 980 can be operably coupled to the controller 988 via an operable connection 985. The operable connection 985 can include one or more of a wireless connection or a physical electrical connection such as wiring.

In some embodiments, the release mechanism 901 can have different configurations than shown in FIG. 9, some configurations can include one or more batteries, timers, or electrical switches as described herein.

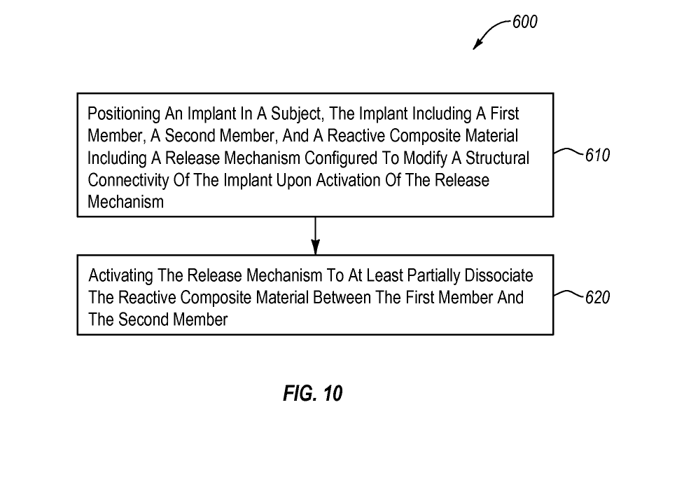

FIG. 10 is a flow diagram of a method 1000 of modifying a modifiable implant according to an embodiment. The method 1000 includes an act 1010 of positioning the implant in or on a subject. The implant can be similar or identical to any modifiable implant disclosed herein. Positioning the implant in or on a subject can include surgical implantation, adhesion, or any other suitable technique for placing an implant in or on a subject. The implant can include a first member similar or identical to any disclosed herein; a second member similar or identical to any disclosed herein; and an RCM including a release mechanism similar or identical to any disclosed herein. The RCM can couple the first and second members together to add structural connectivity in any manner disclosed herein, such as at least the first distal portion of the first member to the second proximal portion of the second member thereby limiting relative movement therebetween.

In addition to or as alternative to positioning an implant, the method 1000 can include locating an implant, such as locating a modifiable implant that was previously implanted or otherwise associated with a subject.

The method 1000 further includes an act 1020 of activating the release mechanism. The act 1020 can include activating the release mechanism to at least partially dissociate the RCM between the first member and the second member. In an embodiment, activating the release mechanism to at least partially dissociate the RCM can include causing a change in the physical state of the reactive composition material, such as at least one of a solid-to-liquid transition, a solid-to-gas transition, a gel-to-liquid transition, a gel-to-gas transition, a foam-to-liquid transition, or a foam-to-gas transition.

In an embodiment, activating the release mechanism to at least partially dissociate the RCM between the first member and the second member includes directing a stimulus such as electromagnetic radiation (e.g., radio frequency radiation or infrared radiation), magnetic force, or sonic vibrations at the release mechanism such as the antenna of the electrical release mechanism. In an embodiment, activating the release mechanism to at least partially dissociate the RCM between the first member and the second member includes releasing the chemical agent and exposing the reactive nanofoil to the chemical agent. In an embodiment, activating release mechanism includes providing an electrical charge to the chemical agent via a capacitor or a radio frequency antenna operably coupled thereto.

In an embodiment, the implant can include more than one RCM and release mechanism. For example, in an embodiment, the implant can include an RCM having a release mechanism and can further include an additional RCM having an additional release mechanism. The additional RCM can couple at least a portion of the first member to at least a portion of the second member to limit movement between the first member and the second member identical or similarly to the RCM. In such embodiments, activating the release mechanism can include activating at least one release mechanism, such as activating the additional release mechanism to at least partially dissociate the additional RCM between the first member and the second member, or activating both release mechanisms either sequentially or simultaneously.

In an embodiment, providing a stimulus to the release mechanism includes directing the radio frequency radiation to the release mechanism such to the antenna. The antenna can collect and convert the radio frequency radiation into electrical charge and deliver the electrical charge to a capacitor operably coupled thereto. In an embodiment, providing a stimulus to the release mechanism includes directing the stimulus to one or more switches in the release mechanism, the one or more switches configured to open or close a circuit or connection to a battery or capacitor as described herein. The one or more switches can include an antenna, the antenna can collect and convert the stimulus into electrical charge and deliver the electrical charge to switches operably coupled thereto to open or close the one or more switches.

In an embodiment, one or more antennas can be configured to receive electromagnetic radiation such as radio frequency radiation, over a selected frequency range. The act of providing a stimulus to the release mechanism can include directing the electromagnetic radiation to the release mechanism at a frequency within the frequency range that the antenna is configured to receive. In an embodiment, the act of providing the stimulus can include providing an encrypted or otherwise encoded stimulus, such as a frequency inverted radio signal or narrow-band radio frequency to an antenna or resonator (e.g., narrow-band resonator) in a release mechanism.

In an embodiment, activation of the release mechanism can be configured to selectively permit lateral motion along an interface between the first member and the second member. In some embodiments, activation of the release mechanism can be configured to selectively permit axial motion along an interface between the first member and the second member (e.g., concentric cylinders or parallel beams).

In an embodiment, an implant can include a plurality of RCM portions (e.g., stacks) at least some of which are integral to (e.g., embedded in the surface or internal to) one or more of the first or second members and spaced from each other a distance. Each of the RCMs can include a portion of a single collective release mechanism or can each include one or more of a plurality of release mechanisms, similar or identical to any release mechanism disclosed herein. Each of the plurality of release mechanisms can be configured to be selectively activated, such as having a unique frequency, wavelength, or encryption associated therewith. In such embodiments, activating the release mechanism to at least partially dissociate the RCM between the first member and the second member can include selectively activating at least some of the plurality of release mechanisms. Selectively activating at least some of the plurality of release mechanisms can alter a compliance or flexibility of the implant collectively or any of the individual members therein. Upon activation of at least some of the plurality of release mechanisms, the structural connectivity of the implant can be altered by compressive, bending, shear, or tensile forces placed on the first or second members. For example, the structural rigidity of a member can be reduced when the volume of space therein is empty after activation of the release mechanism and partially dissociation of the RCM therein. Such an embodiment can have a selectively modifiable structural flexibility and can be susceptible to crushing, bending, or other forces. In some embodiments, such increased compliance or flexibility can be desirable. Accordingly, only some or all of the plurality of release mechanisms can be activated at differing times, or the same time.

In an embodiment, the RCM on one or more members of an implant can be configured to enable the at least a portion of one or more members to release from embedding tissue upon activation of the release mechanism. In an embodiment, the RCM associated with one or more members can be disposed in an interior portion of the one or more members and can be configured to enable at least one of the members to release from an embedding tissue upon activation of the release mechanism. For example, first or second members can be released from the embedding tissue by activating one or more RCMs internal thereto, thereby collapsing or allowing crushing of at least one of the first and second members. Such collapsing or crushing can be effective to provide sufficient clearance for the at least one of the first or second members to be withdrawal from an embedding tissue.

While the examples of the modifiable implants herein are provided in a biological context, non-biological uses are also considered. For example, in a mechanical structure having one or more members joined by a joint, it may be desirable to lock the joint with an RCM similar to those described herein to allow curing time for portions of the structure prior to full use of the joints therein. In other embodiments, installation of a mechanical part can require the part to have a specific conformation during installation but require free movement during use. In an embodiment, the modifiable implant can be a mechanical fastener split into more than one member (e.g., surgical or industrial screw or bolt). For example, the first member can be configured as a threaded shank portion and the second member can be configured as a head portion with an RCM at least partially bonding the first and second members together. The head portion can be dissociable from the threaded portion via actuation of the release mechanism in the RCM. A suitable “implantable” mechanical structure can include an RCM and members substantially similar to any disclosed herein whether used in a biological subject or otherwise.

The reader will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. The reader will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer can opt for a mainly hardware and/or firmware vehicle; alternatively, if flexibility is paramount, the implementer can opt for a mainly software implementation; or, yet again alternatively, the implementer can opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which can vary. The reader will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein can be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, the various embodiments described herein can be implemented, individually and/or collectively, by various types of electro-mechanical systems having a wide range of electrical components such as hardware, software, firmware, or virtually any combination thereof; and a wide range of components that can impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electrical systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context can dictate otherwise.

In a general sense, the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). The subject matter described herein can be implemented in an analog or digital fashion or some combination thereof.

This disclosure has been made with reference to various example embodiments. However, those skilled in the art will recognize that changes and modifications may be made to the embodiments without departing from the scope of the present disclosure. For example, various operational steps, as well as components for carrying out operational steps, may be implemented in alternate ways depending upon the particular application or in consideration of any number of cost functions associated with the operation of the system; e.g., one or more of the steps may be deleted, modified, or combined with other steps.

Additionally, as will be appreciated by one of ordinary skill in the art, principles of the present disclosure, including components, may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create a means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof are intended to cover a non-exclusive inclusion, such that a process, a method, an article, or an apparatus that comprises a list of elements does not include only those elements but may include other elements not expressly listed or inherent to such process, method, system, article, or apparatus.

In an embodiment, the modifiable implants and systems for modifying an implant disclosed herein can be integrated in such a manner that the modifiable implants and systems operate as a unique system configured specifically for the function of structurally or otherwise modifying the implant, and any associated computing devices of the modifiable implants and systems operate as specific use computers for purposes of the claimed system, and not general use computers. In an embodiment, at least one associated computing device of the modifiable implants and systems operate as specific use computers for purposes of the claimed system, and not general use computers. In an embodiment, at least one of the associated computing devices of the modifiable implants and systems are hardwired with a specific ROM to instruct the at least one computing device. In an embodiment, one of skill in the art recognizes that the modifiable implants and systems effects an improvement at least in the technological field of implants.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

In some instances, one or more components can be referred to herein as “configured to.” The reader will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, the recited operations therein can generally be performed in any order. Examples of such alternate orderings can include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

What is claimed is:

1. A modifiable implant, comprising:
a first member;
a second member; and
a reactive composite material coupling at least a portion of the first member to the at least a portion of the second member to limit movement therebetween, the reactive composite material including a release mechanism configured to modify a structural connectivity of the modifiable implant upon activation of the release mechanism.

2. The modifiable implant of claim 1, wherein the release mechanism is configured to change a physical state of the reactive composite material upon activation of the release mechanism.

3. The modifiable implant of claim 2, wherein the release mechanism is configured to change the physical state from a solid to a liquid.

4. The modifiable implant of claim 2, wherein the release mechanism is configured to change the physical state from a solid to a gas.

5. The modifiable implant of claim 2, wherein the reactive composite material includes a phase change material, and wherein the release mechanism is configured to change the physical state of the phase change material.

6. The modifiable implant of claim 1, wherein the release mechanism is configured to initiate a chemical reaction between components of the reactive composite material upon activation of the release mechanism.

7. The modifiable implant of claim 1, wherein the release mechanism is configured to permit lateral motion along an interface between a distal portion of the first member and a proximal portion of the second member upon activation of the release mechanism.

8. The modifiable implant of claim 1, further including an adhesive layer between the reactive composite material and at least one of the first member or the second member.

9. The modifiable implant of claim 1, wherein the reactive composite material includes at least one reactive nanofoil.

10. The modifiable implant of claim 1, wherein reactive composite material includes a plurality of layers including one or more of at least one reactive nanofoil, at least a portion of the release mechanism, or a protective layer.

11. The modifiable implant of claim 1, wherein the release mechanism includes one or more of an electrical release mechanism or a chemical release member.

12. The modifiable implant of claim 1, wherein the release mechanism includes an electrical release mechanism including a circuit having a capacitor and an antenna, and wherein the capacitor is configured to be charged via radio frequency energy, ultrasonic vibrations, or infrared light directed to the antenna and discharged through a resistive material operably coupled to the capacitor.

13. The modifiable implant of claim 1, wherein the release mechanism includes an electrical release mechanism including a circuit having a capacitor and a battery, and wherein the capacitor is configured to be charged via the battery and discharged through a resistive material operably coupled to the capacitor.

14. The modifiable implant of claim 1, wherein the release mechanism includes,
a chemical release member having a chemical agent releasably stored in a compartment therein, the chemical agent composed to degrade the reactive composite material when exposed the reactive composite material; and
a circuit configured to emit an electrical charge effective to rupture the compartment and release the chemical agent therein.

15. The modifiable implant of claim 1, wherein the reactive composite material extends around substantially an entire lateral dimension of each of the first and second members.

16. The modifiable implant of claim 1, further including a joint coupling the first member to the second member.

17. The modifiable implant of claim 16, wherein the reactive composite material limits movement of the joint.

18. The modifiable implant of claim 16, wherein movement of the joint is enhanced following activation of the release mechanism.

19. The modifiable implant of claim 16, wherein the joint is flexible following activation of the release mechanism.

20. The modifiable implant of claim 16, wherein the joint is at least partially rotatable following activation of the release mechanism.

21. The modifiable implant of claim 16, wherein the joint includes a hinge coupling the first member to the second member.

22. The modifiable implant of claim 1, further including:
a third member; and
an additional reactive composite material including an additional release mechanism, the additional reactive composite material coupling at least a portion of the second member to at least a portion of the third member to at least partially limit movement therebetween.

23. The modifiable implant of claim 22, wherein the release mechanism and the additional release mechanism are identically configured, with each of the release mechanism and the additional release mechanism including one or more of an electrical release mechanism or a chemical release member.

24. The modifiable implant of claim 22, wherein the release mechanism and the additional release mechanism are configured differently, with each of the release mechanism and the additional release mechanism including one or more of an electrical release mechanism or a chemical release member.

25. The modifiable implant of claim 1, further including:
a third member;
an additional joint coupling the second member to the third member; and
an additional reactive composite material including an additional release mechanism, the additional reactive composite material coupling at least a portion of the second member to at least a portion of the third member to at least partially limit movement of the additional joint.

26. The modifiable implant of claim 1, wherein the first and second members define a surgical screw, wherein the first member is configured as a threaded portion and the second member is configured as a head portion, and wherein the head portion is dissociable from the threaded portion via actuation of the release mechanism.

27. The modifiable implant of claim 1, wherein the reactive composite material is internal to the first and second members, and wherein activation of the release mechanism alters a structural connectivity of a structure formed by the first and second members.

28. The modifiable implant of claim 1, wherein the reactive composite material includes a plurality of reactive composite material portions each of which is internal to the first and second members and spaced from each other, wherein the release mechanism includes a plurality of release mechanisms each of which is associated with a corresponding one of the plurality of reactive composite material portions, wherein each of the plurality of release mechanisms is configured to be selectively activated.

29. The modifiable implant of claim 1, wherein reactive composite material on at least one of the first member or the second member is configured to enable at least one of the first member or the second member to release from embedding tissue upon activation of the release mechanism.

30. The modifiable implant of claim 29, wherein the reactive composite material is disposed about an exterior portion of at least one of the first or second members and is configured to enable the at least one of the first or second members to release from an embedding tissue upon activation of the release mechanism by at least reducing a lateral dimension of the at least one of the first or second member sufficient to enable withdrawal from an embedding tissue.

31. The modifiable implant of claim 29, wherein the reactive composite material is disposed in an interior portion of at least one of the first or second members and is configured to enable at least one of the first or second members to release from an embedding tissue upon activation of the release mechanism by collapsing at least one of the first and second members effective to provide sufficient clearance for the at least one of the first or second members to be withdrawal from an embedding tissue.

32. A method of modifying an implant, the method comprising:
positioning the implant in a subject, the implant including,
a first member;
a second member;
a reactive composite material including a release mechanism configured to modify a structural connectivity of the implant upon activation of the release mechanism, the reactive composite material coupling at least a portion of the first member to at least a portion of the second member; and
activating the release mechanism to at least partially dissociate the reactive composite material between the first member and the second member.

33. The method of claim 32, wherein the release mechanism includes one or more of an electrical release mechanism or a chemical release member.

34. The method of claim 32, wherein activating the release mechanism to at least partially dissociate the reactive composite material includes causing a change in the physical state of the reactive composition material.

35. The method of claim 32, wherein:
the reactive composite material includes a phase change material;
the release mechanism is configured to change the physical state of the phase change material; and
activating the release mechanism to at least partially dissociate the reactive composite material includes causing a change in the physical state of the phase change material.

36. The method of claim 35, wherein the change in the physical state includes at least one of a solid-to-liquid transition, a solid-to-gas transition, a gel-to-liquid transition, a gel-to-gas transition, a foam-to-liquid transition, or a foam-to-gas transition.

37. The method of claim 32, wherein the release mechanism includes an electrical release mechanism including a resistive material operably coupled to a circuit having a capacitor and an antenna; and
wherein the capacitor is configured to be charged via radio frequency energy directed to the antenna and discharged through the resistive material.

38. The method of claim 37, wherein activating the release mechanism to at least partially dissociate the reactive composite material between the first member and the second member includes directing radio frequency energy at the antenna of the electrical release mechanism.

39. The method of claim 32, wherein the release mechanism includes an electrical release mechanism including a resistive material operably coupled to a circuit having a capacitor and a battery, and wherein the capacitor is configured to be charged via the battery and discharged through the resistive material.

40. The method of claim 32, wherein the release mechanism includes a chemical release member having a chemical agent releasably stored in a compartment therein, the chemical agent composed to degrade the reactive composite material when exposed thereto, the chemical release member including a circuit configured to emit an electrical charge effective to rupture the compartment and release the chemical agent therein.

41. The method of claim 40, wherein activating the release mechanism to at least partially dissociate the reactive composite material between the first member and the second member includes releasing the chemical agent and exposing the reactive nanofoil to the chemical agent.

42. The method of claim 41, wherein activating the release mechanism includes providing an electrical charge to the chemical agent via a capacitor or a radio frequency antenna operably coupled thereto.

43. The method of claim 32, further including:
wherein the implant includes an additional reactive composite material including an additional release mechanism, the additional reactive composite material coupling at least a portion of the first member to at least a portion of the second member to limit movement between the first member and the second member; and
activating the additional release mechanism to at least partially dissociate the additional reactive composite material between the first member and the second member.

44. The method of claim 32, wherein activating the release mechanism to at least partially dissociate the reactive composite material between the first member and the second member includes providing a stimulus to the release mechanism, the stimulus including one or more of radio frequency radiation, infrared radiation, or magnetic force.

45. The method of claim 44, wherein:
the stimulus includes radio frequency radiation; and
providing a stimulus to the release mechanism includes directing radio frequency radiation to an antenna in the release mechanism, the antenna collecting and converting the radio frequency radiation into electrical charge and delivering the electrical charge to a capacitor operably coupled thereto.

46. The method of claim 45, wherein the antenna is configured to receive the radio frequency radiation over a selected frequency range.

47. The method of claim 46, wherein the stimulus is encrypted or encoded.

48. The method of claim 32, wherein activation of the release mechanism is configured to permit lateral motion along an interface between the first member and the second member.

49. The method of claim 32, wherein the implant includes an adhesive layer between the reactive composite material and at least one of the member and the second member.

50. The method of claim 32, wherein the reactive composite includes a plurality of reactive composite material portions each of which is internal to the first and second members and spaced from each other, wherein the release mechanism includes a plurality of release mechanisms each of which is associated with a corresponding one of the plurality of reactive composite material portions; and
activating the release mechanism to at least partially dissociate the reactive composite material between the first member and the second member includes selectively activating at least some of the plurality of release mechanisms.

51. A system for modifying an implant, the system comprising:
an implant including,
a first member;
a second member;
a reactive composite material coupling at least a portion of the first member to at least a portion of the second member to limit movement therebetween, the reactive composite material including a release mechanism; and
a stimulus source configured to provide a stimulus to the release mechanism effective to cause activation thereof.

52. The system of claim 51, wherein reactive composite material includes a plurality of layers including one or more of at least one reactive nanofoil, at least a portion of the release mechanism, a phase change material, or a protective layer.

53. The system of claim 51, wherein the stimulus source includes one or more of an electromagnetic radiation generator, an electromagnetic field generator, or a sonic vibration generator.

54. The system of claim 51, wherein the stimulus source includes an electromagnetic radiation generator configured as a radio frequency generator.

55. The system of claim 51, wherein the release mechanism includes one or more of an electrical release mechanism or a chemical release member.

56. The system of claim 51, wherein the release mechanism includes an electrical release mechanism including a resistive material operably coupled to a circuit having a capacitor and an antenna, and wherein the capacitor is configured to be charged via radio frequency energy directed to the antenna and discharged through the resistive material.

57. The system of claim 51, wherein the release mechanism includes a chemical release member having a chemical agent releasably stored in a compartment therein, the chemical agent composed to degrade the reactive composite material, the chemical release member including a circuit configured to emit an electrical charge effective to rupture the compartment and release the chemical agent therein.

58. The system of claim 51, wherein the reactive composite material extends around an entire lateral dimension of the first and second members.

59. The system of claim 51, further including a joint secured to the first member and the second member, the joint including one or more of an adhesive or a mechanical connection.
